(19) United States
(12) Patent Application Publication
(10) Pub. No.: US 2026/0139210 A1
(43) Pub. Date: May 21, 2026

(54) Modular Bioreactor

(71) Applicant: Evoworks Bio, Inc., Cambridge, MA (US)

(72) Inventors: Ross Beighley, Allston, MA (US); Derek Troiano, Wilmington, NC (US); William Roger Mainwaring-Burton, Cambridge, MA (US)

(21) Appl. No.: 19/410,236

(22) Filed: Dec. 5, 2025

Related U.S. Application Data
(63) Continuation of application No. PCT/US2025/056014, filed on Nov. 18, 2025.
(60) Provisional application No. 63/721,936, filed on Nov. 18, 2024.

(51) Int. Cl. C12M 3/00; C12M 1/00; C12M 1/12; C12M 1/34

(52) U.S. Cl. CPC C12M 23/44; C12M 23/22; C12M 23/28; C12M 23/38; C12M 23/40; C12M 23/48; C12M 25/02; C12M 29/00; C12M 41/34

(57) ABSTRACT

A modular bioreactor system includes a set, or array, of smaller bioreactor chambers, such as laboratory-scale bioreactor chambers, which can be filled and drained together, while also being able to be isolated to permit the multiplexed bioreactor chambers to be used together to create a larger-scale reaction volume, thereby forming a manufacturing-scale bioreactor.

### RELATED APPLICATIONS

This application is a continuation of International Application No. PCT/US2025/056014, filed on Nov. 18, 2025, which designated the United States and published in English, which claims the benefit of U.S. Provisional Application No. 63/721,936, filed on Nov. 18, 2024. The entire teachings of the above applications are incorporated herein by reference.

### BACKGROUND

Commercial operations for the production of everything from liquid fuels and industrial chemicals to pharmaceuticals and food additives are widely transitioning toward biological-based processes (Kähler 2021). Realization of what is often described as the “bioeconomy” will be an enormous undertaking and necessitates a proportionally enormous supply of novel bio-based production platforms; all in addition to the existing demand from the biomedical/pharmaceutical and food/beverage industries, which currently already suffer from significant bottlenecks (Barcelos 2018; El-Chickakli 2016).

Cells and microbial enzymes represent the biological equivalent of factories and industrial catalysts (Liu 2019). However, most wild organisms (and wildly occurring enzymes/biocatalysts) do not possess the characteristics required for use in industrial processes, i.e., ability to produce high titers, rates, and/or yields (TRY values) or other desirable non-native functionalities (Parekh 2000). Moreover, many commercially significant molecules that are produced industrially are not naturally synthesized by biological organisms. Generating cells and biocatalysts that are “unnaturally” productive and/or can catalyze non-naturally occurring reactions requires considerable amounts of time and resources all without knowing whether they can be successfully employed on an industrial scale.

Scale-up, from laboratory bench-top production at small volumes to commercial-scale production, presents scientific and technical uncertainty, thereby causing economic risk. Scale-up also represents a major technical bottleneck since, for example, the otherwise automated and efficient strain development workflow must be interrupted once a suitable candidate strain is generated to pre-screen the candidate for behavior at scale. This is a costly and time-consuming process that, in the end, still does not guarantee success at commercial scale.

The current state of the art for scaling biological product manufacturing processes is lengthy and iterative. Such scaling typically begins with a small volume (e.g., a shake flask). From here, scientists and engineers investigate appropriate parameters for culturing the cells at increasingly larger volumes. Compared to smaller reaction volumes, larger volume reactors may have a heterogeneous distribution of mass (such as nutrients and gases required for cell survival) and heat. These heterogeneous conditions often lead to cell death or significant reductions in, for example, cell growth rates and productivity in producing molecules or proteins of interest.

Addressing cell death/poor performance at scale, e.g., through tuning various parameters, can be prohibitively expensive (potentially millions of dollars per run). Instead, scale up is performed as a stepwise process.

Cells will typically undergo a round of testing at the <1 L scale; the best settings (such as oxygen sparging rate, impeller rotation speed, and nutrient concentration) and procedures are then applied in a larger testing volume (e.g., a 5 L tank). This process is repeated in sequentially larger volumes until the desired scale is achieved. Each round of experimentation may require several (e.g., four) weeks. and due to the size differences among reaction vessels, there is a risk that the growth conditions that worked for the smaller tank will not be suitable at a larger volume, and consequently cells do not survive or production of molecules or proteins of interest is suboptimal. This is considered an unavoidable risk in the state of the art and failures of this sort are common. These failures require teams to return to earlier development stages.

An important consideration is that regulatory agencies, such as the U.S. Food and Drug Administration, require Process Qualification tests that explore the process limits and confirm output in dozens of different situations (such as varying input material qualities, varying ambient conditions such as laboratory humidity, etc.) to ensure the product is functionally the same in all circumstances.

Performing each test at full scale (such as up to about 10,000 L) can be prohibitively expensive and time-consuming, so there is often a need for a “scale-down.” This is a process wherein experimental data is collected to demonstrate that a smaller bioreactor is analogous to the full-scale process, and then the Process Qualification tests are performed using the smaller reactor. Conventional Process Qualification may require several months.

### SUMMARY

In an embodiment according to the invention, a modular bioreactor system includes an array of bioreactor chambers, such as laboratory-scale bioreactor chambers (such as with a volume between about 50 mL and about 5 L), which can be filled and drained together, while also being able to be isolated to permit the multiplexed bioreactor chambers to be used together to create a larger-scale reaction volume, thereby forming a manufacturing-scale bioreactor. Further related modular bioreactor systems are taught herein.

Embodiments disclosed herein are small modular bioreactors that, when arranged in an array of the bioreactor system, have a collective volume comparable to a large bioreactor but mass, heat, and gas distribution dynamics of a small reactor. In other words, individual bioreactor chambers can be integrated together to achieve the desired production scale. Each individual chamber is a small-volume bioreactor, which ensures that even at large production volumes, the cells are being cultured within small scale conditions (such as homogeneous, or near-homogeneous, distribution of nutrients, heat, and gases).

The modular bioreactor system can enable a faster time to scale up for bio-derived products, such as vaccines, and may improve scale-up times by months. In microbial production strain development processes, multiple stages, including different production volumes, must be investigated, due to changing conditions within bioreactors as their volume increases. This variability is the result of the different chemical and fluid dynamics in larger volumes. By maintaining fluid conditions constant as net volume increases, an embodiment according to the invention allows skipping of intermediate volume testing steps required in strain development; and can also provide an associated reduction of scale-up risk, since the intermediate steps are fraught with risks, such as cells dying in a short period of time and/or an inability to produce, or suboptimal production of, the molecules or proteins of interest. Further advantages are taught herein.

In one embodiment, a bioreactor system comprises a chamber support assembly configured to mount a support rail, the support rail being configured to support a set of bioreactor chamber nodes. A fluid manifold comprises a fill line fluid connection configured to permit filling of the set of bioreactor chamber nodes, and a drain line fluid connection configured to permit draining of the set of bioreactor chamber nodes. A chamber interface assembly comprises an optical guide configured to aim an optical fiber at an optical access window of a bioreactor chamber node of the set of bioreactor chamber nodes.

In further, related embodiments, the chamber support assembly may be configured to slidingly receive the support rail. The bioreactor system may further comprise the support rail, the support rail comprising a bracket configured to receive a top cap of a bioreactor chamber node. The bioreactor system may comprise the set of bioreactor chamber nodes. A support rail front plate may comprise at least one sample tubing port. The support rail front plate may comprise at least one sample tubing port for each bioreactor chamber node of the set of bioreactor chamber nodes. A pair of parallel support rails may be configured to support the at least one set of the plurality of bioreactor chamber nodes. A support rail front plate may comprise at least one sample tubing port, the support rail front plate being mounted across the pair of parallel support rails. The bioreactor system may further comprise a scaffold frame configured to receive the chamber support assembly. The scaffold frame may be configured to mechanically mount the bioreactor system within an incubator. The scaffold frame may comprise a perforated airflow panel.

In other related embodiments, the bioreactor system may further comprise the chamber support assembly, the chamber support assembly comprising a bioreactor chamber set lock with a notch configured to locate the set of bioreactor chamber nodes in position after the set is inserted into the chamber support assembly. The chamber interface assembly may further comprise a guide rail configured to fit with an alignment rail of a bioreactor chamber node of the set of bioreactor chamber nodes. The chamber interface assembly may further comprise a valve actuator configured to rotate a plurality of valves to pinch closed base tubing, the base tubing providing fluid connection between at least two bioreactor chamber nodes of the set of bioreactor chamber nodes. The valve actuator may be configured to rotate all of the plurality of valves to pinch closed the base tubing for all of the bioreactor chamber nodes of the set of bioreactor chamber nodes. The chamber interface assembly may further comprise an optical fiber for each bioreactor chamber node of the set of bioreactor chamber nodes; or the chamber interface assembly may further comprise more than one optical fiber for each bioreactor chamber node of the set of bioreactor chamber nodes.

In further related embodiments, the chamber interface assembly may comprise a linear actuator configured to position the optical guide relative to the optical access window of the bioreactor chamber node. The chamber interface assembly may further comprise a strain gauge configured to determine a weight of a bioreactor chamber node of the plurality of bioreactor chamber nodes. The set of bioreactor chamber nodes may be configured to be suspended from the support rail, and the strain gauge may be configured to sense a weight added to the bioreactor chamber node via additional load on the base of the bioreactor chamber node when the weight is added to the bioreactor chamber node. The set of bioreactor chamber nodes may be suspended from the support rail in a mechanically compressed state. An optical multiplexer module may be configured for optical sensing of the bioreactor chamber nodes of the set of bioreactor chamber nodes. The optical multiplexer module may be configured to restrict sensing to one bioreactor chamber node of the plurality of bioreactor chamber nodes at a given time, and may be configured to change the one bioreactor chamber node to be sensed. The optical multiplexer module may comprise a linear stage to position an optical sensor relative to at least one optical fiber. At least one optical fiber may be configured to perform optical sensing of at least one bioreactor chamber node of the plurality of bioreactor chamber nodes. The chamber interface assembly may further comprise: a guide rail configured to fit with an alignment rail of a bioreactor chamber node of the set of bioreactor chamber nodes; a valve actuator configured to rotate a plurality of valves to pinch closed base tubing, the base tubing providing fluid connection between at least two bioreactor chamber nodes of the set of bioreactor chamber nodes; and a strain gauge configured to determine a weight of the bioreactor chamber node of the plurality of bioreactor chamber nodes.

In other related embodiments, the bioreactor system may further comprise a bioreactor chamber node of the set of bioreactor chamber nodes. The bioreactor chamber node comprises a flexible, oxygen-permeable membrane configured to contain a biotic or abiotic material for a bioreaction. A rigid top cap may be mounted to a top portion of the flexible, oxygen-permeable membrane, the rigid top cap configured to mount to the support rail. A rigid bottom cap may be mounted to a bottom portion of the flexible, oxygen-permeable membrane, the rigid bottom cap comprising at least one fluid access port. An optical access window may be configured to permit optical monitoring of the bioreaction by an optical fiber. The bioreactor chamber node may be configured to be suspended from the support rail, such as in a mechanically compressed state. The at least one fluid access port may comprise a fill port and a dispense port. The bioreactor chamber nodes may further comprise: a fill connection barb, in fluid connection with the fill port, configured to provide fluid connection to fill base tubing of the bioreactor system; and a dispense connection barb in fluid connection with the dispense port, configured to provide fluid connection to dispense base tubing of the bioreactor system. An alignment rail may be configured to align the node with a guide rail of the chamber interface assembly of the bioreactor system. The bioreactor chamber node may be a disposable module that is configured to be used once and then disposed of.

In further related embodiments, the chamber interface assembly may further comprise a plurality of valves configured to restrict fluid flow to or from the bioreactor chamber nodes. The fill line fluid connection and a plurality of fluid access ports of the bioreactor chamber nodes may be connected to permit filling of all bioreactor chamber nodes of the set of bioreactor chamber nodes in a single filling. The fill line fluid connection and a plurality of fluid access ports of each of the bioreactor chamber nodes of the bioreactor system may be connected to permit filling of all of the plurality of bioreactor chamber nodes of the bioreactor system in a single filling. A controlled environment containment may surround the bioreactor system. The controlled environment containment may comprise a gas surrounding the bioreactor system. The controlled environment containment may comprise an incubator. The bioreactor system may comprise sensor system configured to receive an optical signal from an optical fiber. The bioreactor system may, based on input from the optical fiber, measure dissolved oxygen in the bioreactor chamber nodes, measure dissolved carbon dioxide in the bioreactor chamber nodes, measure pH in the bioreactor chamber nodes, measure fill level in the bioreactor chamber nodes, and/or perform Raman spectroscopy on the bioreactor chamber nodes. A system fluid manifold may distribute a single fluidic fill path to a plurality of sets of the bioreactor chamber nodes. A system fluid manifold may collect a single fluidic drain path from a plurality of sets of the bioreactor chamber nodes.

In another embodiment, a bioreactor system comprises a chamber support assembly configured to mount a support rail, the support rail configured to support a set of bioreactor chamber nodes. A fluid manifold comprises a fill line fluid connection configured to permit filling of the set of bioreactor chamber nodes, and a drain line fluid connection configured to permit draining of the set of bioreactor chamber nodes. The bioreactor system comprises a bioreactor chamber node of the set of bioreactor chamber nodes. The bioreactor chamber node comprises a flexible, oxygen-permeable membrane configured to contain a biotic or abiotic material for a bioreaction. A rigid top cap is mounted to a top portion of the flexible, oxygen-permeable membrane, the rigid top cap configured to mount to the support rail. A rigid bottom cap is mounted to a bottom portion of the flexible, oxygen-permeable membrane, the rigid bottom cap comprising at least one fluid access port. An optical access window is configured to permit optical monitoring of the bioreaction by an optical fiber.

In further, related embodiments, the bioreactor chamber node may further comprise an alignment rail configured to align the bioreactor chamber node with a guide rail of a chamber interface assembly of the bioreactor system. The bioreactor chamber node may be configured to be suspended from the support rail, such as in a mechanically compressed state. The at least one fluid access port may comprise a fill port and a dispense port. A fill connection barb, in fluid connection with the fill port, may be configured to provide fluid connection to fill base tubing of the bioreactor system; and a dispense connection barb in fluid connection with the dispense port, may be configured to provide fluid connection to dispense base tubing of the bioreactor system.

In other related embodiments, a plurality of valves may be configured to restrict fluid flow to or from the plurality of bioreactor chamber nodes. The fill line fluid connection and a plurality of fluid access ports of the bioreactor chamber nodes may be connected to permit filling of all bioreactor chamber nodes of the set of bioreactor chamber nodes in a single filling. The fill line fluid connection and a plurality of fluid access ports of the bioreactor chamber nodes may be connected to permit filling of all of the plurality of bioreactor chamber nodes of the bioreactor system in a single filling. The bioreactor chamber node may be a disposable module that is configured to be used once and then disposed of. A controlled environment containment may surround the bioreactor system. The controlled environment containment may comprise a gas surrounding the bioreactor system. The controlled environment containment may comprise an incubator. The bioreactor system may comprise a sensor system configured to receive an optical signal from an optical fiber. Based on input from the optical fiber, the sensor system may measure dissolved oxygen in the bioreactor chamber nodes, measure dissolved carbon dioxide in the bioreactor chamber nodes, measure pH in the bioreactor chamber nodes, measure fill level in the bioreactor chamber nodes, and/or perform Raman spectroscopy on the bioreactor chamber nodes. A system fluid manifold may distribute a single fluidic fill path to a plurality of sets of the bioreactor chamber nodes. A system fluid manifold may collect a single fluidic drain path from a plurality of sets of the bioreactor chamber nodes.

In another embodiment, a bioreactor system comprises a mounting frame configured to support a single bioreactor chamber node, the mounting frame configured to receive a support rail that is configured to mount to the single bioreactor chamber node, and the mounting frame being further configured to mechanically mount the bioreactor system within an incubator. A bottom chamber mount comprises a guide rail configured to fit with an alignment rail of the single bioreactor chamber node. The bioreactor system comprises a lock mechanism for the single bioreactor chamber node. An optical guide is configured to aim an optical fiber at an optical access window of the single bioreactor chamber node.

In further, related embodiments, the bioreactor system may further comprise the support rail, the support rail being slidingly engaged with the mounting frame. The support rail may comprise a bracket configured to receive a rigid top cap of the single bioreactor chamber node. A support rail front plate may comprise at least one sample tubing port. A pair of parallel support rails may be configured to support the single bioreactor chamber node. A support rail front plate may comprise at least one sample tubing port, the support rail front plate being mounted across the pair of parallel support rails. The mounting frame may comprise at least one open side to permit air flow around the single bioreactor chamber node. The bioreactor system may further comprise the single bioreactor chamber node, the single bioreactor chamber node comprising: a flexible, oxygen-permeable membrane configured to contain a biotic or abiotic material for a bioreaction; a rigid top cap mounted to a top portion of the flexible, oxygen-permeable membrane, the rigid top cap configured to mount to the support rail; a rigid bottom cap mounted to a bottom portion of the flexible, oxygen-permeable membrane; and an optical access window configured to permit optical monitoring of the bioreaction by the optical fiber. The rigid bottom cap may further comprise an alignment rail configured to align the bioreactor chamber node with the guide rail. The rigid top cap may further comprise an opening for a closure cap; and may further comprise a sample tubing barb configured to permit fluid connection with an interior of the bioreactor chamber node defined by the flexible, oxygen-permeable membrane, the rigid top cap, and the rigid bottom cap. A dip tube may extend within the interior of the flexible, oxygen-permeable membrane. The flexible, oxygen-permeable membrane may comprise at least one of: silicone; a silicone derivative; polypropylene, polystyrene, polytetrafluoroethylene (PTFE), and polyethylene; and may comprise a thickness of less than about 5 mil. The flexible, oxygen-permeable membrane may comprise polypropylene comprising a thickness of less than about 5 mil; and may comprise a volume of less than about 500 ml. The rigid top cap and the rigid bottom cap may each be substantially circular in cross-section. The single bioreactor chamber node may be a disposable module that is configured to be used once and then disposed of.

In other related embodiments, the lock mechanism may comprise a lower locking arm configured to constrain a rigid bottom cap of the single bioreactor chamber node. The lock mechanism may further comprise an actuator of the lower locking arm; and may further comprise an upper locking arm configured to constrain a rigid top cap of the single bioreactor chamber node. The mounting frame may further comprise a detent that engages with the support rail. An optical slide element may be configured to move the optical guide relative to the optical access window of the single bioreactor chamber node. The bioreactor system may further comprise the optical fiber. The bioreactor system may further comprise a bracket configured to mechanically mount the bioreactor system within an incubator.

In another embodiment, a bioreactor chamber assembly for use in a bioreactor system comprises a support rail supporting a set of a plurality of bioreactor chamber nodes, the plurality of bioreactor chamber nodes being configured to be suspended from the support rail. Each bioreactor chamber node of the plurality of bioreactor chamber nodes comprises a flexible, oxygen-permeable membrane configured to contain a biotic or abiotic material for a bioreaction; a rigid top cap mounted to a top portion of the flexible, oxygen-permeable membrane, the rigid top cap configured to mount to the support rail; a rigid bottom cap mounted to a bottom portion of the flexible, oxygen-permeable membrane, the rigid bottom cap comprising at least one fluid access port; and an optical access window configured to permit optical monitoring of the bioreaction by an optical fiber.

In further, related embodiments the plurality of bioreactor chamber nodes may be configured to be suspended from the support rail, such as in a mechanically compressed state. The at least one fluid access port may comprise a fill port and a dispense port. Each bioreactor chamber node of the plurality of bioreactor chamber nodes may further comprise: a fill connection barb, in fluid connection with the fill port, configured to provide fluid connection to fill base tubing of the bioreactor system; and a dispense connection barb in fluid connection with the dispense port, configured to provide fluid connection to dispense base tubing of the bioreactor system. Each bioreactor chamber node of the plurality of bioreactor chamber nodes may further comprise an alignment rail configured to align the node with a guide rail of a chamber interface assembly of the bioreactor system. The bioreactor chamber assembly may be a disposable module that is configured to be used once and then disposed of.

In another embodiment, a bioreactor chamber node for a bioreactor system comprises a flexible, oxygen-permeable membrane configured to contain a biotic or abiotic material for a bioreaction. A rigid top cap is mounted to a top portion of the flexible, oxygen-permeable membrane, the rigid top cap configured to mount to a support rail of a bioreactor chamber assembly. A rigid bottom cap is mounted to a bottom portion of the flexible, oxygen-permeable membrane, the rigid bottom cap comprising at least one fluid access port. An optical access window is configured to provide optical monitoring of the bioreaction by an optical fiber.

In further, related embodiments, the bioreactor chamber node may be configured to be suspended from the support rail, such as in a mechanically compressed state. The at least one fluid access port may comprise a fill port and a dispense port. The bioreactor chamber node may further comprise: a fill connection barb, in fluid connection with the fill port, configured to provide fluid connection to fill base tubing of the bioreactor system; and a dispense connection barb in fluid connection with the dispense port, configured to provide fluid connection to dispense base tubing of the bioreactor system. An alignment rail may be configured to align the bioreactor chamber node with a guide rail of a chamber interface assembly of the bioreactor system. The rigid bottom cap may comprise an interlock configured to connect to another bioreactor chamber node. The optical access window may comprise an optical access window mounted to the flexible, oxygen-permeable membrane. The optical access window may comprise a transparent region of the rigid bottom cap; and the transparent region may comprise at least one of: acrylic, glass, quartz, polystyrene, polycarbonate, sapphire, polypropylene and polyethylene. The transparent region may comprise at least one of acrylic or glass. The optical access window may comprise an elongated transparent region of the rigid bottom cap. The bioreactor chamber node may comprise an alignment rail configured to align the bioreactor chamber node with a guide rail of a chamber interface assembly of the bioreactor system, and the elongated transparent region of the optical access window may extend substantially parallel to the alignment rail.

In other related embodiments, the at least one fluid access port may comprise a fill port and a dispense port, the bioreactor chamber node further comprising a fill connection barb, in fluid connection with the fill port, configured to provide fluid connection to fill base tubing of the bioreactor system, and a dispense connection barb in fluid connection with the dispense port, configured to provide fluid connection to dispense base tubing of the bioreactor system, and the fill connection barb and the dispense connection barb may comprise elongated fluidic barbs extending substantially parallel to the alignment rail. The at least one fluid access port may comprise a fill port and a dispense port, the bioreactor chamber node further comprising a fill connection barb, in fluid connection with the fill port, configured to provide fluid connection to fill base tubing of the bioreactor system, and a dispense connection barb in fluid connection with the dispense port, configured to provide fluid connection to dispense base tubing of the bioreactor system, and the bioreactor chamber node may comprises an alignment rail configured to align the node with a guide rail of a chamber interface assembly of the bioreactor system, and the fill connection barb and the dispense connection barb may comprise elongated fluidic barbs extending substantially parallel to the alignment rail.

In further related embodiments, the bioreactor chamber node may further comprise a top clamp ring configured to create a seal around a periphery of the rigid top cap, thereby mounting the rigid top cap to the top portion of the flexible, oxygen-permeable membrane; and a bottom clamp ring configured to create a seal around a periphery of the rigid bottom cap, thereby mounting the rigid bottom cap to the bottom portion of the flexible, oxygen-permeable membrane. The rigid top cap may comprise a clip configured to mount to a bracket of the support rail. The rigid top cap may further comprise an opening for a closure cap. The bioreactor chamber node may further comprise the closure cap. The closure cap may comprise a vent. The rigid top cap may further comprise a sample tubing barb configured to permit fluid connection with an interior of the bioreactor chamber defined by the flexible, oxygen-permeable membrane, the rigid top cap, and the rigid bottom cap. The rigid top cap may further comprise a sample tubing clip. A dip tube extending within the interior of the flexible, oxygen-permeable membrane. The flexible, oxygen-permeable membrane may comprise at least one of: silicone; a silicone derivative; polypropylene, polystyrene, polytetrafluoroethylene (PTFE), and polyethylene. The flexible, oxygen-permeable membrane may comprise a thickness of less than about 5 mil; and may comprise polypropylene comprising a thickness of less than about 5 mil. The flexible, oxygen-permeable membrane may be collapsible; and may be sealed to the rigid top cap and sealed to the rigid bottom cap; and may comprise a volume of less than about 500 ml. The rigid top cap and the rigid bottom cap may each be substantially circular in cross-section. The bioreactor chamber node may be a disposable module that is configured to be used once and then disposed of. The bioreactor chamber node may comprise a feature configured to promote mixing in the bioreactor chamber node; which may comprise at least one of: a baffle, a tangential ramp, a radial ramp, a cross-section transition, a membrane twist, and a corrugated membrane.

In another embodiment, a bioreactor chamber comprises at least one array of bioreactor chambers, a plurality of the bioreactor chambers each configured to contain at least one of a biotic material for a bioreaction and an abiotic material for the bioreaction. A plurality of bioreactor chambers of the at least one array serve both (i) to each permit flow of the at least one of the biotic material and the abiotic material for the bioreaction to another bioreactor chamber of the at least one array during at least one of filling and draining of the plurality of bioreactor chambers, and (ii) to incubate the bioreaction. At least one flow control valve isolates each of the bioreactor chambers of the plurality of bioreactor chambers from each other during incubation of the bioreaction. A product drain is configured to drain a product of the bioreactor system from the array.

In further related embodiments, the bioreactor system may further comprise at least one array comprising the bioreactor chambers, the plurality of the bioreactor chambers each being configured to contain the at least one of a biotic material for the bioreaction and the abiotic material for the bioreaction and each comprising: (i) a first fluid interconnection configured to permit filling of each of the plurality of bioreactor chambers in the array through the first fluid interconnection into the bioreactor chamber when a flow control valve of the first fluid interconnection is open to flow from another bioreactor chamber in the array; (ii) a second fluid interconnection configured to permit draining from each bioreactor chamber of the plurality of bioreactor chambers in the array though the second fluid interconnection into another bioreactor chamber in the array when a flow control valve of the second fluid interconnection is open to flow; and (iii) a supply fluid interconnection configured to permit flow of a supply of at least one of a gas and a bioreaction culture media component into each bioreactor chamber of the plurality of bioreactor chambers in the array. The product drain may be configured to drain a product of the bioreactor system from the array when at least one second fluid interconnection of at least one bioreactor chamber of the array is open.

In other related embodiments, the bioreactor system may further comprise a scaffold supporting the at least one array comprising the bioreactor chambers; and a supply channel supported by the scaffold and configured to supply at least one bioreactor chamber of the array of bioreactor chambers. A fluid manifold may be configured to supply at least one of: a bioreaction culture media, a gas, and a bioreaction culture media component to at least one bioreactor chamber of the array of bioreactor chambers. At least one bioreactor chamber of the array of bioreactor chambers may comprise a consumable container; may comprise a flexible container; and may comprise an oxygen permeable membrane. The bioreactor system may comprise a scaffold, and the scaffold may comprise at least one sensor configured to sense a status of a bioreaction occurring in at least one bioreaction chamber of the array. The sensor may comprise an optical sensor. The bioreactor system may comprise an interconnecting fluid tube between a first bioreactor chamber and a second bioreactor chamber in the array. The at least one flow control valve to isolate each of the bioreactor chambers may comprise a flow control valve configured to open and close fluid flow through the interconnecting fluid tube. A valve may permit direct fluid flow between a first bioreactor chamber and a second bioreactor chamber of the array. Each bioreactor chamber of the array may comprise a volume of between about 50 mL and about 50 L, and a plurality of bioreactor chambers of the bioreactor system together may comprise a reaction volume of at least about 100 L.

In other related embodiments, a flow control unit may be configured to control filling of the array in a series flow through the plurality of bioreactor chambers of the array. The flow control unit may be further configured to control closing of at least one fluid interconnection of the array to isolate each bioreactor chamber of the array such that each such bioreactor chamber is configured to incubate the bioreaction. The flow control unit may be further configured to drain a product of a bioreaction from at least one bioreactor chamber of the array. The flow control unit may be further configured to maintain substantially consistent reaction conditions within each bioreactor chamber of the array. A plurality of arrays may be arranged in a parallel flow arrangement in the bioreactor system. A controlled environment containment may surround the at least one array comprising bioreactor chambers. A volume between the at least one array and the controlled environment containment may comprise a gas. The bioreactor system may be configured to maintain substantially consistent reaction conditions within each bioreactor chamber of the array. Each bioreactor chamber of the at least one array may comprise a volume of between about 50 mL and about 50 L; and may comprise a volume of between about 50 mL and about 5 L. The bioreactor system may not have an impeller. The at least one of the biotic material and the abiotic material for the bioreaction may comprise at least one of a bioreaction culture media and a gas.

In several embodiments, each bioreactor chamber of the at least one array may comprise a volume of between about 1 mL and about 50 mL, and a plurality of bioreactor chambers of the bioreactor system together may comprise a reaction volume of at least about 100 mL. In yet another embodiment, each bioreactor chamber of the at least one array may comprise a volume of between about 50 L and about 500 L; and may comprise a volume of between about 5 L and about 250 L, and a plurality of bioreactor chambers of the bioreactor system together may comprise a reaction volume of at least about 1,000 L. In yet another embodiment, each bioreactor chamber of the at least one array may comprise a volume of between about 500 L and about 5,000 L; and may comprise a volume of between about 500 L and about 2,500 L, and a plurality of bioreactor chambers of the bioreactor system together may comprise a reaction volume of at least about 10,000 L.

In another embodiment, a method of culturing cells comprises a) filling a plurality of bioreactor chambers arranged in the at least one array of any of a bioreactor system taught herein; b) isolating a plurality of the bioreactor chambers when each bioreactor chamber of the array is filled; c) culturing cells within the plurality of bioreactor chambers so filled; and d) opening valves to drain a product of the culturing of the cells in the one or more bioreactor chambers of the at least one array such that the bioreactor chambers drain via flow through a plurality of bioreactor chambers of the array.

In another embodiment, a method of scaling up biomanufacturing comprises a) performing a bioreaction in a bioreactor comprising a volume of between about 50 mL and about 50 L; and b) subsequently culturing cells in an array of bioreactor chambers each comprising a volume of between about 50 mL and about 50 L arranged in a bioreactor system taught herein comprising the array of bioreactor chambers each comprising the volume of between about 50 mL and about 50 L in the at least one array of the bioreactor system taught herein.

In a related embodiment, the method may comprise performing the bioreaction in a bioreactor comprising a volume of between about 50 mL and about 5 L; and subsequently culturing the cells may comprise culturing the cells in an array of bioreactor chambers each comprising a volume of between about 50 mL and about 5 L.

In another embodiment, a bioreactor system comprises at least one flow tube connecting a plurality of bioreactor chambers in an array, the flow tubes conducting fluid flows between the bioreactor chambers. A plurality of fluid inlets are distributed along the at least one flow tubes, the plurality of fluid inlets providing valved control between the flow tubes and the plurality of bioreactor chambers. Each bioreactor chamber connects to a fluid inlet of the plurality of fluid inlets as a passive flow element.

In further, related embodiments, a dip tube, aseptic sampling chamber, or port may be used for the extraction of fluid from each bioreactor chamber or addition of gas or nutrients to each bioreactor chamber. The dip tube, aseptic sampling chamber, or port may be used for the extraction of fluid, and the fluid is to be used for sample analysis.

In another embodiment, a bioreactor chamber is configured to contain at least one of a biotic material for a bioreaction and an abiotic material for a bioreaction, wherein the bioreactor chamber is configured (i) to permit flow of the at least one of the biotic material and the abiotic material for the bioreaction to another such bioreactor chamber when formed into at least one array of such bioreactor chambers during at least one of filling and draining of a plurality of such bioreactor chambers, and (ii) to incubate the bioreaction, the array of such bioreactor chambers being arranged in a bioreactor system taught herein.

In a further, related embodiment, the bioreactor chamber may comprise: (i) a first fluid interconnection configured to permit filling of the bioreactor chamber through the first fluid interconnection into the bioreactor chamber when a flow control valve of the first fluid interconnection is open to flow from another bioreactor chamber in the at least one array; (ii) a second fluid interconnection configured to permit draining from the bioreactor chamber though the second fluid interconnection into another bioreactor chamber in the array when a flow control valve of the second fluid interconnection is open to flow; and (iii) a supply fluid interconnection configured to permit flow of a supply of at least one of a gas and a bioreaction culture media component into the bioreactor chamber.

In another embodiment, a method of performing a bioreaction, such as culturing cells, comprises: a) filling a set of bioreactor chamber nodes arranged in a bioreactor system, the filling of the set of bioreactor chamber nodes being performed in a single filling step; b) isolating the set of bioreactor chamber nodes when each bioreactor chamber node is filled; c) performing a bioreaction within the isolated set of bioreactor chamber nodes so filled; and d) opening at least one valve to drain a product of the bioreaction in the set of bioreactor chamber nodes.

In further, related embodiments, the bioreactor system may comprise any of the bioreactor systems taught herein. The method may comprise filling a plurality of sets of bioreactor chamber nodes of the bioreactor system in a single filling. A controlled environment containment may surround the bioreactor system. The controlled environment containment may comprise a gas surrounding the bioreactor system. The controlled environment containment may comprise an incubator. The method may further comprise receiving an optical signal from an optical fiber. The method may further comprise, based on input from the optical fiber, measuring dissolved oxygen in the bioreactor chamber node, measuring dissolved carbon dioxide in the bioreactor chamber node, measuring pH in the bioreactor chamber node, measuring fill level in the bioreactor chamber nodes, and/or performing Raman spectroscopy on the plurality of bioreactor chamber nodes. An optical multiplexer may be used to select between performing one or more of measuring dissolved oxygen in the bioreactor chamber node, measuring dissolved carbon dioxide in the bioreactor chamber node, measuring pH in the bioreactor chamber node, measuring fill level in the bioreactor chamber nodes, and performing Raman spectroscopy on the plurality of bioreactor chamber nodes. A weight measurement may be performed on the set of bioreactor chamber nodes. The single filling step may be performed upon a fill command signaled from a user. The method may comprise filling a level of a bioreactor system, the level comprising a plurality of sets of bioreactor chamber nodes. A weight measurement may be performed to detect filling of the sets of bioreactor chamber nodes of the level of the bioreactor system. The method may further comprise initiating a filling of another level of the bioreactor system, the another level comprising a further plurality of sets of bioreactor chamber nodes. When each bioreactor chamber node is filled, the method may comprise performing sensing on the set of bioreactor chamber nodes using an optical fiber.

In another embodiment, a bioreactor system comprises: a) a plurality of bioreactor chamber nodes arranged in a bioreactor system, the plurality of bioreactor chamber nodes being configured to be filled in a single filling step; b) the plurality of bioreactor chamber nodes being configured to be isolated when at least each bioreactor chamber node of the single filling step is filled, and to perform a bioreaction within the isolated plurality of bioreactor chamber nodes so filled; and c) at least one valve configured to be opened to drain a product of the bioreaction in the plurality of bioreactor chamber nodes.

In further, related embodiments, the bioreactor system may further comprise a controlled environment containment surrounding the bioreactor system. The controlled environment containment may comprise a gas surrounding the bioreactor system. The controlled environment containment may comprise an incubator.

In another embodiment, a method of scaling biomanufacturing comprises: a) performing a bioreaction in a bioreactor chamber node comprising a volume of between about 50 mL and about 50 L; and b) subsequently culturing cells in a plurality of bioreactor chamber nodes each comprising a volume of between about 50 mL and about 50 L arranged in a bioreactor system taught herein comprising the plurality of bioreactor chamber nodes each comprising the volume of between about 50 mL and about 50 L in the bioreactor system taught herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram showing a bioreactor system in accordance with an embodiment of the invention, which includes at least one vertical array including bioreactor chambers.

FIG. 2 is a schematic diagram of a bioreactor system including multiple parallel vertical arrays of bioreactor chambers, in accordance with an embodiment of the invention.

FIG. 3 is a schematic diagram of a bioreactor system including multiple parallel vertical arrays of bioreactor chambers arranged on a scaffold, in accordance with an embodiment of the invention.

FIG. 4 is a schematic diagram of a bioreactor system with a controlled environment containment, in accordance with an embodiment of the invention.

FIGS. 5A-5E are schematic diagrams of a bioreactor system including a direct connection valve, in accordance with an embodiment of the invention.

FIG. 6 is a schematic diagram of a bioreactor system using a “fuel rail” arrangement of flow tubes connecting bioreactor chambers, in accordance with an embodiment of the invention.

FIGS. 7A-B are schematic diagrams of a bioreactor system without (FIG. 7A) and with (FIG. 7B) bioreactor chamber nodes, in accordance with an embodiment of the invention.

FIGS. 8A-B are schematic diagrams of a bioreactor chamber assembly (perspective views from above and below, respectively), in accordance with an embodiment of the invention.

FIGS. 9A-B are schematic diagrams of a chamber support assembly and chamber interface assembly (perspective views of each from above and below, respectively), in accordance with an embodiment of the invention.

FIGS. 10A-B are schematic diagrams of the scaffold frame (perspective views from above and below, respectively), in accordance with an embodiment of the invention.

FIG. 11 is a schematic diagram of a bioreactor chamber assembly, including a support rail, in accordance with an embodiment of the invention.

FIGS. 12A-B are schematic diagrams of a chamber interface assembly (perspective views from two different vantage points), in accordance with an embodiment of the invention.

FIGS. 13A-B are schematic diagrams of a chamber support assembly (perspective views from two different vantage points), each in accordance with an embodiment of the invention.

FIG. 14A-C are schematic diagrams showing the chamber interface assembly, in accordance with an embodiment of the invention. FIG. 14A is a top view, omitting the cosmetic covers to illustrate operation of the valving. FIG. 14B is a bottom view illustrating the operation on the optical guides. FIG. 14C is a perspective view from below, omitting the optical guides to illustrate operation of a weight measurement module.

FIG. 15 is a schematic diagram illustrating use of an optical multiplexer, in accordance with an embodiment of the invention.

FIGS. 16A-B are schematic diagrams of a bioreactor chamber node (perspective views from above and below, respectively), in accordance with an embodiment of the invention.

FIG. 17 is a schematic diagram showing bioreactor chamber nodes in a mechanically compressed state, in accordance with an embodiment of the invention.

FIGS. 18A-C are schematic diagrams illustrating connections to the bioreactor chamber nodes, in accordance with an embodiment of the invention.

FIGS. 19A-B are schematic diagrams showing an embodiment of the bioreactor chamber node that includes a single fluid access port, in accordance with an embodiment of the invention.

FIG. 20 is a schematic diagram showing an embodiment of a bioreactor chamber node in which the optical access window is mounted to the flexible, oxygen-permeable membrane, for example in a position where it can be optically interrogated from the side of the bioreactor chamber node, in accordance with an embodiment of the invention.

FIG. 21 is a schematic diagram of a bioreactor system for a single bioreactor chamber node, in accordance with an embodiment of the invention.

FIGS. 22A-C are schematic diagrams of the bioreactor system of FIG. 21, showing a single bioreactor chamber node installed in the system, in accordance with an embodiment of the invention.

FIG. 23 is a schematic diagram showing a bottom view of the bioreactor system of FIGS. 21 and 22A-C, in accordance with an embodiment of the invention.

FIG. 24 is a system architecture diagram for a bioreactor system, in accordance with an embodiment of the invention.

FIGS. 25A-25B are a process flow diagram for use of a bioreactor system, in accordance with an embodiment of the invention.

FIGS. 26A-G are schematic diagrams illustrating features for mixing in bioreactor chamber nodes, in accordance with an embodiment of the invention.

FIG. 27 is a schematic block diagram of a controls module in a bioreactor system in accordance with an embodiment of the invention.

### DETAILED DESCRIPTION

A description of example embodiments follows.

In an embodiment according to the invention, a modular bioreactor system includes an array of smaller bioreactor chambers, such as laboratory-scale bioreactor chambers, which can be filled and drained together, while also being able to be isolated to permit the multiplexed bioreactor chambers to be used together to create a larger-scale reaction volume, thereby forming a manufacturing-scale bioreactor. By replicating the conditions of a small bioreactor many times over, a large volume bioreactor system can be made that operates with internal conditions identical to a single small bioreactor.

As used herein, “bioreaction” refers to any type of biofermentation, bioprocessing, or bioproduction, and includes such bioreactions occurring by microbes, bacteria, fungi, plant cells, and mammalian cells, as well as acellular enzymatic reactions. A “bioreactor” or “bioreactor system” refers to a device or apparatus, or system, within which the bioreaction occurs, and may include the supporting structures for the supply of nutrients and collection of products generated by the bioreaction. While therapeutic products and applications are discussed herein, embodiments can also be used for non-therapeutic products and applications, for example in the microbial manufacture of biofuels.

An important issue in large bioreactors is heterogeneous conditions within the reactor. The contents of bioreactors may be poorly mixed because it is often physically impossible to mix them sufficiently enough to achieve homogeneous conditions, which may be more readily achieved at small scale. Achieving a more homogeneous distribution within a large bioreactor requires increasing the impeller rates, which requires additional energy (which can be prohibitive at larger volumes) and increases shear stress on the cells, which can adversely affect cell performance (such as by decreased viability, growth rate, productivity, etc.).

Several embodiments described herein are characterized by the absence of an impeller, which is unnecessary for small reaction volumes.

Embodiments disclosed herein are small modular bioreactors that, when arranged in an array of the bioreactor system, have a collective volume comparable to a large bioreactor but mass, heat, and gas distribution dynamics of a small reactor. In other words, individual bioreactor chambers can be integrated together to achieve the desired production scale. Each individual chamber is a small-volume bioreactor, which ensures that even at large production volumes, the cells are being cultured within small scale conditions (such as homogeneous, or near-homogeneous, distribution of nutrients, heat, and gases).

The modular bioreactor system can enable a faster time to scale up for bio-derived products, such as vaccines, and may improve scale-up times by months. In microbial production strain development processes, multiple stages, including different production volumes, must be investigated, due to changing conditions within bioreactors as their volume increases. This variability is the result of the different chemical and fluid dynamics in larger volumes. By maintaining fluid conditions constant as net volume increases, an embodiment according to the invention allows skipping of intermediate volume testing steps required in strain development; and can also provide an associated reduction of scale-up risk, since the intermediate steps are fraught with risks, such as cells dying in a short period of time and/or an inability to produce, or suboptimal production of, the molecules or proteins of interest.

Embodiments can also avoid the need for “scale-down” justification work when executing on process qualification and can thereby significantly reduce regulatory burden (potentially by multiple months).

Further, embodiments can reduce the cost of capital equipment, and associated real estate for capital equipment, because removing the need for intermediate volume bioreactors also removes the need for their capital equipment on the way to production scale.

Embodiments can reduce the impact of single-infection events by allowing chamber isolation and removal. Conventionally, on the order of 10% of all cultures are lost to infection, with impacts often ranging upwards of $100 million.

Embodiments address the conventional inability to easily fill and pool multiple lab-scale volumes; and reduce the high costs of maintaining a large catalog of slightly increasing bioreactor sizes.

Modular volumes can be used as both incubation volumes and “flow-through”; that is, each bioreactor chamber in an array serves as both the vehicle for volume to pass through and a chamber for incubation. Among other advantages, this reduces the number of tubes and lines in the bioreactor system.

Embodiments can also provide equally distributed oxygenation and agitation, which is typically required to ensure homogenous growth conditions. If feed lines and bleed lines are used, scaffolds can support the feed lines and bleed lines that run to and from each bioreaction chamber in an array, thereby again reducing complexity.

Embodiments therefore facilitate filling and draining (which can be performed aseptically) of multiple networked laboratory-scale bioreactors or bioreactor microenvironments, which achieve consistent, predictable results at larger scales. By using an array of bioreactors (e.g., having a size less than 1L), embodiments can reduce time to market (often on the order of magnitude of months), save money, and reduce technical and scientific risk. An additional benefit is the capability of mitigating contamination by isolating a single bioreactor. Additionally, there may be less variation within a given “batch.” Here, it is noted that a “batch” for a modular bioreactor system taught herein can refer to a production batch that is created from combining together the products of the entire plurality of bioreactor chambers that are included within the modular bioreactor system to produce a single product “batch” run, even though each bioreactor chamber may be isolated from other small scale bioreactor chambers within the modular bioreactor system during production of the overall “batch,” and in that sense each smaller scale bioreactor chamber is producing its own sub-batch within the overall “batch” of the modular bioreactor system; whereas, conventional bioreactors typically produce a single “batch” in a single, larger, manufacturing scale bioreactor.

Compared to prior workflows, yields using modular bioreactor systems taught herein can be more consistent when scaling up to larger volumes. Importantly, modular bioreactor systems described herein considerably lower the barrier to entry for new drugs to enter early trials and enable life-saving biologics to be developed significantly faster and cheaper than the current state of the art.

In several embodiments, each bioreactor chamber (node) (e.g., of a set of bioreactor chamber nodes, or of an array of bioreactor chambers) can comprise a volume of between about 1 mL and about 50 mL, and a plurality of bioreactor chambers (nodes) of the bioreactor system together may comprise a reaction volume of at least about 100 mL. In yet another embodiment, each bioreactor chamber (node) may comprise a volume of between about 50 L and about 500 L; and may comprise a volume of between about 5 L and about 250 L, and a plurality of bioreactor chambers (e.g., of a set of bioreactor chamber nodes, or of any array of bioreactor chamber nodes, or of a bioreactor system) collectively may comprise a reaction volume of at least about 1,000 L. In yet another embodiment, each bioreactor chamber comprises a volume of between about 500 L and about 5,000 L; and may comprise a volume of between about 500 L and about 2,500 L, and a plurality of bioreactor chambers (e.g., a set or an array) of the bioreactor system together may comprise a reaction volume of at least about 10,000 L.

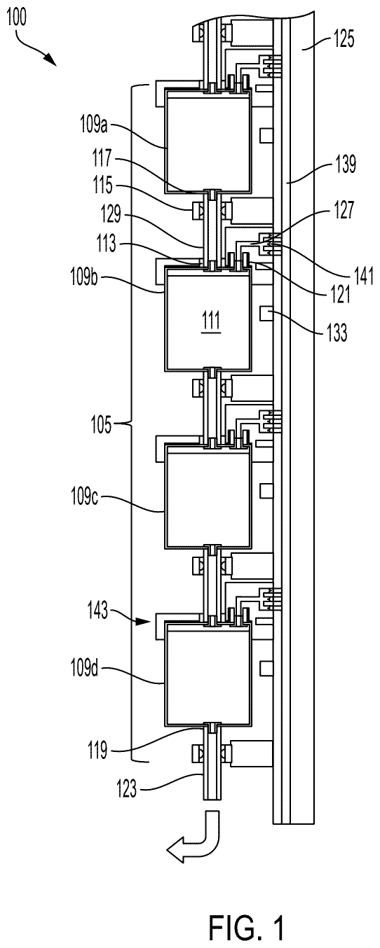

FIG. 1 is a schematic diagram showing a bioreactor system 100 in accordance with an embodiment of the invention, which includes at least one vertical array 105 including bioreactor chambers 109a-d. Here, an “array” of bioreactor chambers includes at least two bioreactor chambers in flow connection with each other (which flow connection can be capable of being isolated, such as during incubation of the bioreaction), but can include any plural number of bioreactor chambers. The number of bioreactor chambers in an array can, for example, be varied depending on the production volume desired. A plurality of the bioreactor chambers 109a-d each include a bioreactor chamber 109a, 109b, 109c, 109d (noting that, here, vertical array 105 includes chambers 109a, 109b, 109c, 109d in vertical series from top to bottom). The bioreactor chambers 109a-109d are configured to contain culture media 111. A top fluid interconnection 113 (here shown on bioreactor chamber 109b) is configured to permit filling of each of the plurality of bioreactor chambers 109a-d in the vertical array 105 through the top fluid interconnection 113 into the bioreactor chamber 109b when a flow control valve 115 between the top fluid interconnection 113 and bottom fluid interconnection 117 is open to flow from another bioreactor chamber 109a above the bioreactor chamber 109b in the vertical array 105. (Here, it is noted that a top fluid interconnection of a topmost bioreactor chamber, not shown, in an array 105 will have no bioreactor chamber above it but will instead function as a fill inlet for the entire vertical array 109a, 109b, 109c, 109d). A bottom fluid interconnection 117 (shown for chamber 109a) is configured to permit draining from each bioreactor chamber 109a-d of the plurality of bioreactor chambers 109a-d in the vertical array 105 though the bottom fluid interconnection 117 into a bioreactor chamber 109b below the bioreactor chamber 109a in the vertical array 105 when a flow control valve 115 of the bottom fluid interconnection 117 is open to flow. A supply fluid interconnection 121 is configured to permit flow of a supply of at least one of a gas and a culture media component into each bioreactor chamber 109a-109d of the plurality of bioreactor chambers 109a-109d in the vertical array 105. It is noted that gases can, for example, alternatively (or in addition) be introduced at a bottom of a bioreactor chamber 109a-109d, to permit the gas to bubble up through the media in the chamber; or can be introduced at one or more other locations on the bioreactor chamber 109a-109d, such as at the top of the chamber. A product drain 123 is configured to drain a product of the bioreactor system 100 from the vertical array 105 when at least one bottom fluid interconnection 119 (shown here for chamber 109d) of at least one bioreactor chamber 109d of the vertical array 105 is open. Although FIG. 1 illustrates a vertical array 105, the array 105 need not be vertical, for example if a pump is used. The bioreactor chambers 109a-d may comprise (i) a first fluid interconnection 113 configured to permit filling of the bioreactor chamber (such as 109b) through the first fluid interconnection 113 into the bioreactor chamber 109b when a flow control valve 115 of the first fluid interconnection 113 is open to flow from another bioreactor chamber (such as 109a) in the at least one array; (ii) a second fluid interconnection 117 configured to permit draining from the bioreactor chamber (such as 109a) though the second fluid interconnection into another bioreactor chamber (such as 109b) in the array when a flow control valve 115 of the second fluid interconnection is open to flow; and (iii) a supply fluid interconnection 121 configured to permit flow of a supply of at least one of a gas and a bioreaction culture media component into the bioreactor chamber 109a-d.

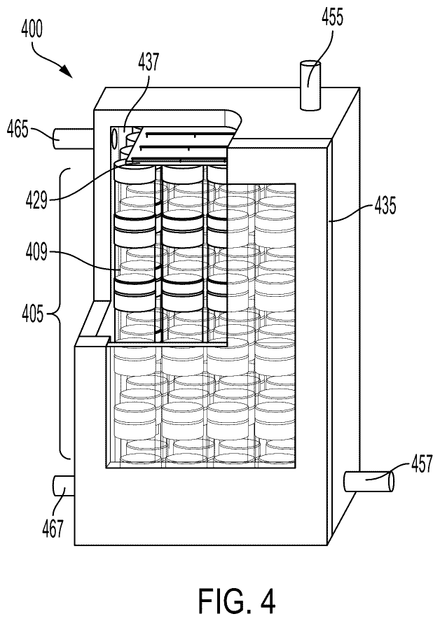

The bioreactor system 100 can further include a scaffold 125 supporting the vertical array 105 of bioreactor chambers 109a-d. A supply channel 127 can be supported by the scaffold 125 and configured to supply at least one of the gas and the culture media component through the supply fluid interconnection 121 into the bioreactor chambers (shown here for 109b) of the vertical array 105. The bioreactor system 100 can further include a fluid manifold 429 (shown analogously in the bioreactor system 400 of the embodiment of FIG. 4) configured to supply culture media or a component thereof and/or gas to the bioreactor chambers 109a-109d (see FIG. 1) of the vertical array 105. For example, such a fluid manifold 429 can be in fluid connection with one or more supply channels 127 of one or more vertical arrays 105 in the bioreactor system 100, for example to supply channels 127 of multiple parallel vertical arrays 105 in the bioreactor system 100. The bioreactor chambers 109a-109d can be or include one or more of a consumable container, a flexible container, and an oxygen-permeable membrane. For example, the wall of each bioreactor chamber 109a-109d can be or include an oxygen-permeable membrane. The scaffold 125 can include at least one sensor 133, such as an optical sensor or temperature sensor, configured to sense a status of a bioreaction occurring in at least one of the bioreaction chambers 109a-d of the vertical array 105. Other methods of reaction monitoring can be used; for example, sampling ports (not shown) can also be used to monitor reactions (for example by aseptic sampling), and on-board sensors inside the bioreactor chambers 109a-109d can be used for sensing in manners other than optical sensing (such as temperature sensors). An interconnecting fluid tube 129 (which can be flexible) can be between the bottom fluid interconnection (such as 117) of a first bioreactor chamber 109a above a second bioreactor chamber 109b in the vertical array, and the top fluid interconnection 113 of the second bioreactor chamber 109b. At least one of the top fluid interconnection 113 and the bottom fluid interconnection 117 may include a flow control valve 115 configured to open and close fluid flow through the interconnecting fluid tube 129.

The bioreactor system 100 can include a frame, such as scaffold 125, which can be reusable and which allows installation of each bioreactor chamber 109a-d in the array 105. Typically, the bioreactor chambers 109a-d are intended for single-use. Each of these bioreactor chambers 109a-d network with the bioreactor chamber above and below it (such as the bioreactor chamber 109b networking with that for bioreactor chamber 109a above it and bioreactor chamber 109c below it), allowing seamless flow-through without the need for extra tubing/consumables. This reduces complexity and points of failure. Additionally, once the system is incubating, each bioreactor chamber 109a-d can be closed off from the others through valves, such as flow control valves 115. Consequently, failure of one bioreactor chamber 109a-d is easily isolated, and the failed bioreactor chamber 109a-d can be replaced without compromising the rest of the bioreactor system 100. The scaffold 125 can include internal fluid distribution channels 139. Seals or flow control valves 141 can be used in supply channels 127 to control or seal the flow of liquids, gases, and/or solids (including cells or solid substrates) to and from the bioreactor chambers 109a-109d. A rigid frame 143 can assist to hold the bioreactor chambers 109a-109d to the scaffold 125 and associated supply channels.

Each bioreactor chamber 109a-d of the vertical array 105 can be a laboratory scale bioreactor, such as with a volume between about 50 mL and about 5 L, or even larger, such as for bioproduction of commodities, for example with a volume of between about 50 mL and about 50 L; and a plurality of bioreactor chambers 109 a-d can together comprise a reaction volume equal to that of a manufacturing scale bioreactor, such as up to about 10,000 L. A flow control unit (not shown) can be configured to control filling of the vertical array 105 in a gravitationally downward direction in vertical series through the plurality of bioreactor chambers 109a-109d of the vertical array 105. The flow control unit can be further configured to control closing of at least one top fluid interconnection 113 and bottom fluid interconnection 117 of the vertical array 105 to isolate each bioreactor chamber 109a-d such that each bioreactor chamber 109a-d incubates a bioreaction; and can be further configured to drain a product of a bioreaction from at least one bioreactor chamber 109a-d of the vertical array 105. The flow control unit can be further configured to maintain substantially consistent reaction conditions within each bioreactor chamber 109a-d of the vertical array 105 by supplying flow through each supply fluid interconnection 121 of each bioreactor chamber of the plurality of bioreactor chambers 109a-109d in the vertical array 105.

Although embodiments are discussed herein with reference to a vertical array such as shown in FIG. 1, an array 105 need not be vertical. Although a vertical array 105 may provide the advantage of being able to be filled by series flow using draining downwards from bioreactor chambers that are gravitationally above others, that need not be the case. For example, pumping may be used to allow series flow through of a non-vertical array of bioreactor chambers. More generally, a bioreactor system can include an array 105 of bioreactor chambers 109a-d, where a plurality of the bioreactor chambers 109a-d are each configured to contain at least one of a biotic material for a bioreaction and an abiotic material for the bioreaction (such as, for example, bioreaction culture media, gas, or any other biotic or abiotic materials needed for the bioreaction). A plurality of bioreactor chambers of the at least one array 105 can serve both (i) to each permit flow, such as serial flow, of the at least one of the biotic material and the abiotic material for the bioreaction to another bioreactor chamber of the at least one array during at least one of filling and draining of the plurality of bioreactor chambers, and (ii) to incubate the bioreaction. At least one mechanism (such as flow control valves 115) can be used to isolate each of the bioreactor chambers 109a-d from each other during incubation of the bioreaction; and a product drain 123 can be configured to drain a product of the bioreactor system from the array 105. The bioreactor system 100 (as well as other bioreactor systems taught herein, including those of FIGS. 1-6) can be characterized by not having an impeller, which is unnecessary for small reaction volumes.

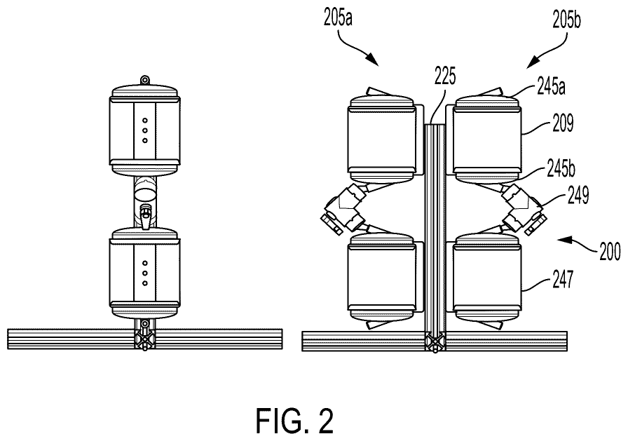

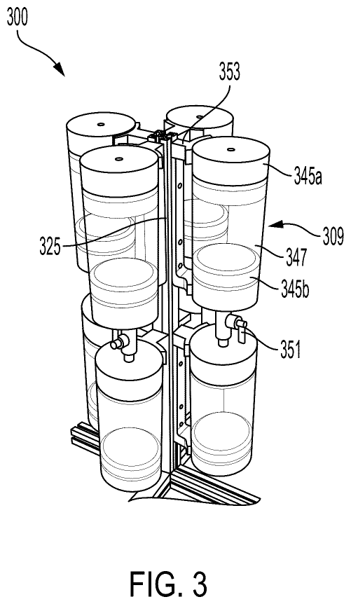

With reference to the embodiment of FIG. 2, a bioreactor system 200 can include a plurality of vertical arrays 205a, 205b arranged in a parallel flow arrangement in the bioreactor system (two vertical arrays are shown in FIG. 2; four vertical arrays are shown in the embodiment of FIG. 3; it will be appreciated that different numbers can be used, such as in the embodiment of FIG. 4). In FIG. 2, there is also shown an alternative type of bioreactor chamber 209 with a rigid upper lid 245a, a rigid lower lid 245b, a flexible semipermeable barrier 247, and an angled flow control valve 249 for vertical series flow. In this example, four bioreactor chambers are shown mounted on a central scaffold 225 in two vertical arrays, but it will be appreciated that different numbers can be used.

With reference to the embodiment of FIG. 3, a bioreactor system 300 is shown that can, for example, be used for small batch runs, using small incubators. A central scaffold 325 supports eight bioreactor chambers in four vertical arrays using chamber support frames 353. Each bioreactor chamber 309 has a rigid upper lid 345a and lower lid 345b, and a flexible membrane 347. Connecting valves 351 connect bioreactor chambers of each vertical array.

With reference to the embodiment of FIG. 4, the bioreactor system 400 can further include a controlled environment containment 435 (here shown cut-away for clarity) surrounding the at least one vertical array 405 including bioreactor chambers 409. Such a bioreactor system 400 can, for example, be a dedicated incubator design for medium scale. The controlled environment containment 435 can be a temperature-and humidity-controlled environment, such as an incubator. The bioreactor system 400 includes a bulk inflow 455 into an upper fluid manifold 429 and a bulk outflow 457 from a lower fluid manifold (not shown; similar to upper fluid manifold 429), as well as a gas inlet port 465 and a gas outlet port 467. A volume 437 between the at least one vertical array 405 and the controlled environment containment 435 can include a gas. The bioreactor system 400 can be configured to maintain substantially consistent reaction conditions within each bioreactor chamber 409 of the vertical array 405. Each bioreactor chamber 409 of the at least one vertical array 405 can, for example, comprise a volume of between about 50 mL and about 5 L, or for example of between about 50 mL and about 50 L.

In another embodiment according to the invention, a method of culturing cells includes filling a plurality of bioreactor chambers arranged in the at least one array 105 of any of the bioreactor systems taught herein; isolating a plurality of the bioreactor chambers when the array is filled; culturing cells within the plurality of bioreactor chambers so filled; and opening valves 123 to drain a product of the culturing of the cells in the one or more bioreactor chambers of the at least one array 105 such that the bioreactor chambers drain via flow, such as serial flow, through a plurality of bioreactor chambers of the array.

In a further embodiment according to the invention, a method of scaling up biomanufacturing includes performing a bioreaction in a bioreactor comprising a volume of between about 50 mL and about 50 L, more particularly such as between about 50 mL and about 5 L and subsequently culturing cells in an array of bioreactor chambers 109 a-d each comprising such a volume, arranged in any of the bioreactor systems taught herein that include the array of bioreactor chambers 109a-d comprising such a volume in the at least one array 105 of the bioreactor system.

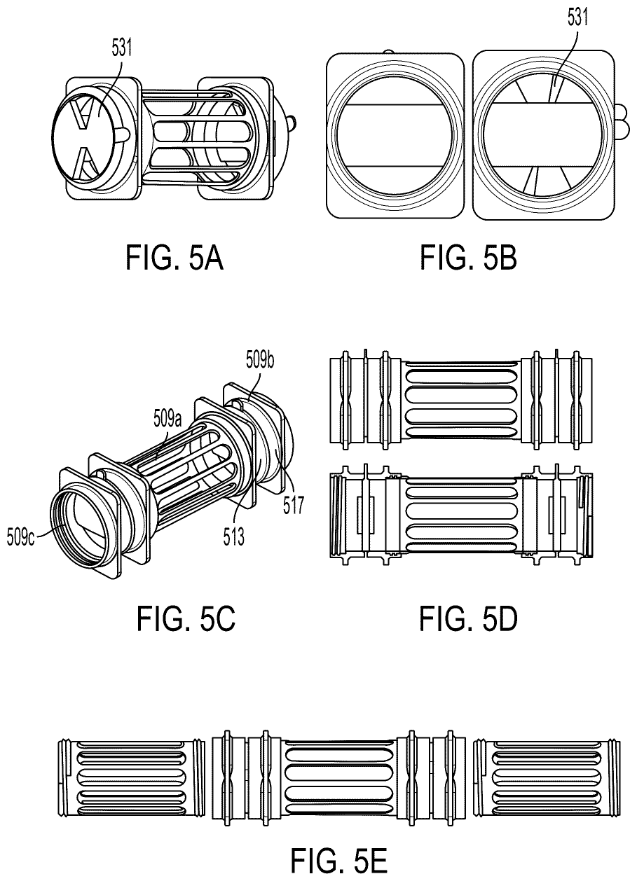

FIGS. 5A-5E are schematic diagrams of a bioreactor system including a direct connection valve 531, in accordance with an embodiment of the invention. FIG. 5A shows a view of an individual bioreactor chamber with open connecting valves 531; FIG. 5B shows a view of an individual chamber from above with closed (left) and open (right) connecting valves 531; FIG. 5C shows a view of an individual chamber 509a integrated with two other chambers 509b, 509c (only connectors shown for other chambers); FIG. 5D shows a side profile of individual chamber integrated with two other chambers (only connectors shown for other chambers) as viewed from exterior (above) and interior (below) of chamber; and FIG. 5E shows a side profile of three integrated chambers (reactor chambers for peripheral chambers shown detached from connectors).

In the embodiment of FIGS. 5A-5E, at least one of the top fluid interconnection 513 and the bottom fluid interconnection 517 may include a valve 531, such as a gate valve, permitting direct fluid flow between the bottom fluid interconnection 517 of a first bioreactor chamber 509b above a second bioreactor chamber 509a in the vertical array, and the top fluid interconnection 513 of the second bioreactor chamber 509a.

Embodiments include a system for connecting multiple small bioreactors 109a-109d (see FIG. 1) such that they can each act as an individual reaction chamber, with substantially the same fluidic, gas, agitation, thermal and therefore biological behavior as one another, while being interconnected for the purpose of rapid filling or dispensing of the contents of all interconnected bioreactors. A single chamber 109a-109d can, for example, have a volume of between about 50 mL and about 5 L, or between about 50 mL and about 50 L, and can be connected to additional chambers to bring the total volume of fluid to any volume up to, for example, 10,000 L.

These bioreactor chambers 409 (see FIG. 4) can be contained within a controlled environment 435 (incubator) which maintains temperature and other characteristics such as agitation (shaking), airflow and gas concentration to ensure that growth conditions remain substantially constant across every chamber, with the possible inclusion of fans for airflow, heating and chilling for temperature control within the environment 435 and a shake plate for agitation. The bioreactor chambers 409 can be configured in accordance with any of the bioreactor chambers disclosed herein, such as bioreactor chambers 109a-d, 209, 309, 509a-c, 609a.

Tubing 129 (see FIG. 1) can connect the fluid chambers to one another for filling and dispensing, with a means 127 for the evacuation and addition of gas to the chambers as the fluid enters and leaves respectively. Multiple chains 205a, 205b (see FIG. 2) of chambers can be connected to coalesce their flows to enable simultaneous filling or emptying of multiple chains.

The chambers (such as any of the bioreactor chambers disclosed herein, such as 109a-d, 209, 309, 409, 509a-c, 609a), can be arranged in a regular pattern, where they are stacked on top of one another and/or arranged adjacent in width and/or depth. In some embodiments, the chambers can be hung from a rigid scaffold (see FIG. 3).

The chambers 109a-109d themselves can be rigid, flexible or partially flexible in nature.

If a scaffold 125 is used, it can hang from one or more walls of the incubator 435 or rest on the base of the incubator. This scaffold 125 can be rigid, flexible or partially flexible.

Secondary fluid paths 139, 127 can run along the scaffold or be otherwise supported for the delivery of additional nutrients, gas supply or other materials to affect the reaction during, before or after the growth within the chamber. These fluid paths can be permanent parts of the scaffold or can be disposable elements which are connected to the chambers.

Control of flow between chambers and between the chambers and secondary fluid paths can be controlled by valves 115, 119. These valves can be part of the chamber or part of the scaffold within the incubator.

Sensors 133 can be present on one or multiple of the chambers for the purpose of monitoring gas concentrations, temperature, turbidity, pH or other factors which can affect the growth within the chamber or inform the status of the reaction. Data gathered on chambers within the incubator can be used to extrapolate the properties within chambers within the same incubator which have no monitoring.

The chambers 109a-109d themselves can be disposable once their use is complete, or the chambers can contain a disposable lining or disposable elements which permit reuse of some elements of the chamber but disposal of others.

The chambers 109a-109d themselves can be loaded individually into the incubator 435 or can be loaded in batches to hasten the process. Some additional support structure or tool can be used for loading of the chambers into the incubator and removal once the reaction is complete.

The chambers and the incubator can be sterilized using gamma, ethylene oxide, ultraviolet or some other suitable method before use, and parts of the system which are not disposed of can be re-sterilized for further uses.

In an alternative embodiment, a bioreactor system can include a large bioreaction volume that is filled, and then active elements break the volume into smaller identical bioreactor volumes during incubation, such as in a tank that segments its larger volume into smaller ones for use as bioreactor chambers. More generally, a modular bioreactor system can include a larger environment that is filled with bioreaction biotic and/or abiotic materials and then isolated into smaller environments, which become bioreactor chambers for incubation of the bioreaction.

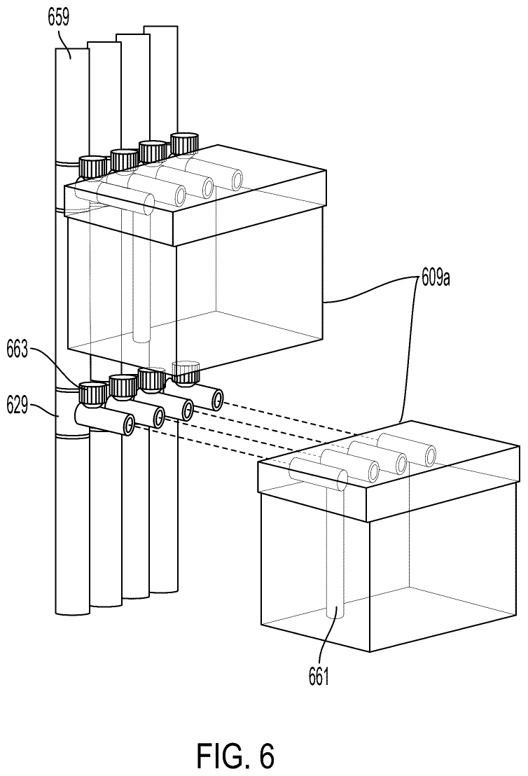

FIG. 6 is a schematic diagram of a bioreactor system using a “fuel rail” arrangement of flow tubes connecting modular bioreactor chambers 609a, in accordance with an embodiment of the invention. In this embodiment, a network of flow tubes 659 conduct fluid flows, such as cell culture media components, gas flows, or liquid flows (or all of them), between bioreactor chambers 609a of the bioreactor chambers. The flow tubes 659 perform fluid circulation (such as circulation of media, filling of the bioreactor chambers 609a, and gas transfer). Parallel fluid inlets 629 are distributed along these flow tubes 659, providing valved control via valve controls 663 (which may be automated) between the flow tubes 659 and the bioreactor chambers 609a, thereby providing a dosing mechanism to the bioreactor chambers 609a. The bioreactor chambers 609a connect to the fluid inlets 629 as passive flow elements. Dip tubes 661, aseptic sampling chambers, or ports, can be used for the extraction of fluid from the bioreactor chamber 609a or addition of gas or nutrients to the bioreactor chamber 609a. For example, the dip tubes 661, aseptic sampling chambers, or ports, can be used to extract fluid that is to be used for sample analysis.

In an embodiment according to the invention, the environment at production-scale can be the same as the environment at laboratory-scale, but with more of the laboratory-scale environments being multiplexed together. This makes the conventional process development workflow described above obsolete, and it reduces inherent risk in scaling, reduces months, and reduces the capital expenses of maintaining multiple different bioreactors in a clean biologics environment.

Advantages of embodiments can be illustrated by considering the concept of “pooling.” If an early-development laboratory has a 500 mL shake flask of their cells and is looking to make more product for an early test (before clinical trials or even animal testing), they have some choices. The laboratory could purchase a 5L tank and do the extensive scale-up work described above. Alternatively, they could grow up ten of the 500 mL flasks, then pour them all into one pool. This has significantly reduced biological risk associated with it because it is not changing the volume in each flask, but it does have other risks: for example, the laboratory might run out of room, their incubators may have subtle heat gradients in them, or they may not be able to pool the flasks aseptically.

By contrast, embodiments described herein can provide a standalone chamber (for example, of about 500 mL) that can be networked aseptically, in series and/or in parallel, to culture a larger volume, for example up to 2000 L (4000×500 mL) with identical performance at any scale. The resulting economy of scale allows the use of many networked chambers without increasing the complexity or cost of operation above the current standard. This large parallel-culture reactor functions like a tank: a user will pour in cells and media which will be routed to the many (such as, for example, as 4,000 or more) separate bioreactor chambers within it. These separate chambers then seal off from one another, physically and thermally. Each has its own exposure to the common air environment via gas-permeable membranes. The system incubates in this state. Then, when it is time to empty the parallel-culture reactor, the valves open back up and allow all of the individual chambers to empty into one channel.

The common environment is air that is temperature-and gas-controlled (e.g., for oxygen and CO₂) with sufficient circulation to maintain homogeneity within. The system (such as any of the systems taught herein, including the systems of FIGS. 1-6) can sit atop an agitator, thereby ensuring consistent agitation.

Using an embodiment according to the invention, a laboratory can go from one chamber at lab scale to multiple at any final volume with consistent performance, and without requiring scale-up or scale-down tests.

In rendering the conventional scale-up workflow obsolete, users can realize advantages such as: a reduction in process development costs; a multiple month reduction in overall timelines; a reduction of biological and regulatory risk; reduced volumetric effects, which means that batch variation will be inherently reduced; reduced real-estate due to the removal of the mid-scale bioreactors; a modular, isolated design that can section off contamination events so the remainder of a batch can be salvaged, noting that these events conventionally occur in up to 10% of all bioproduction batches and can account for hundreds-of-millions in losses. Such advantages can reduce the impediments to new drug development, catalyze new business for industry, and remove barriers surrounding early testing for drugs.

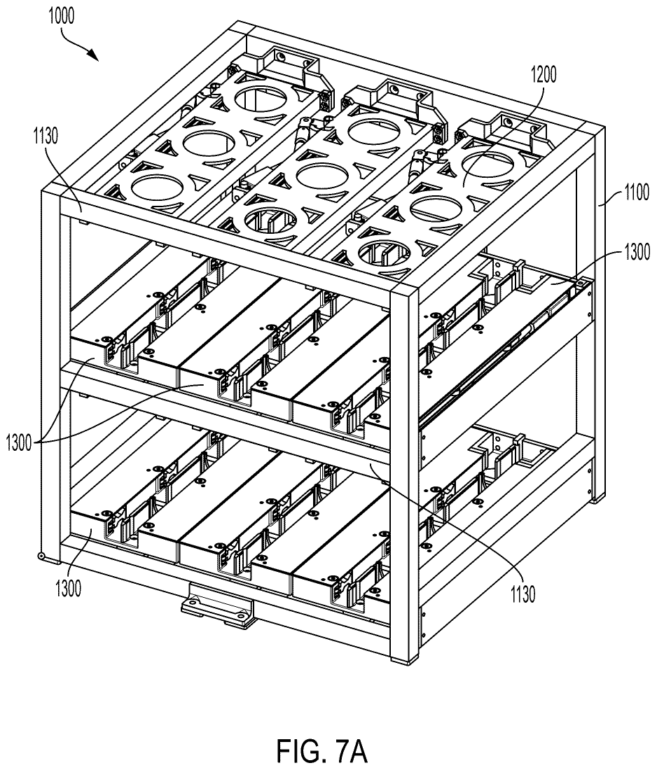

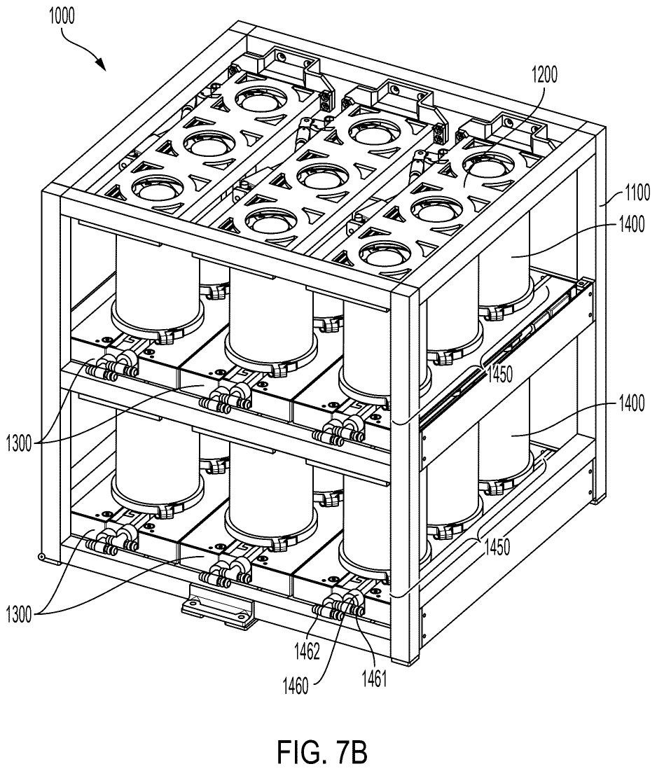

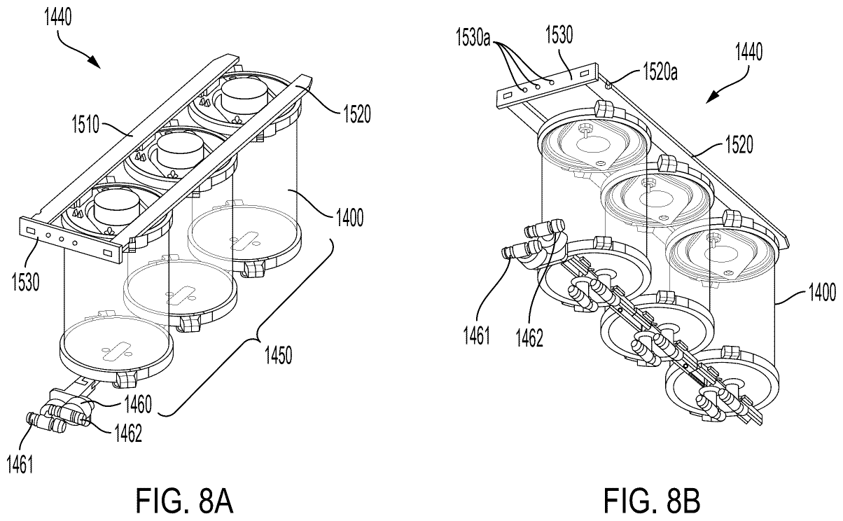

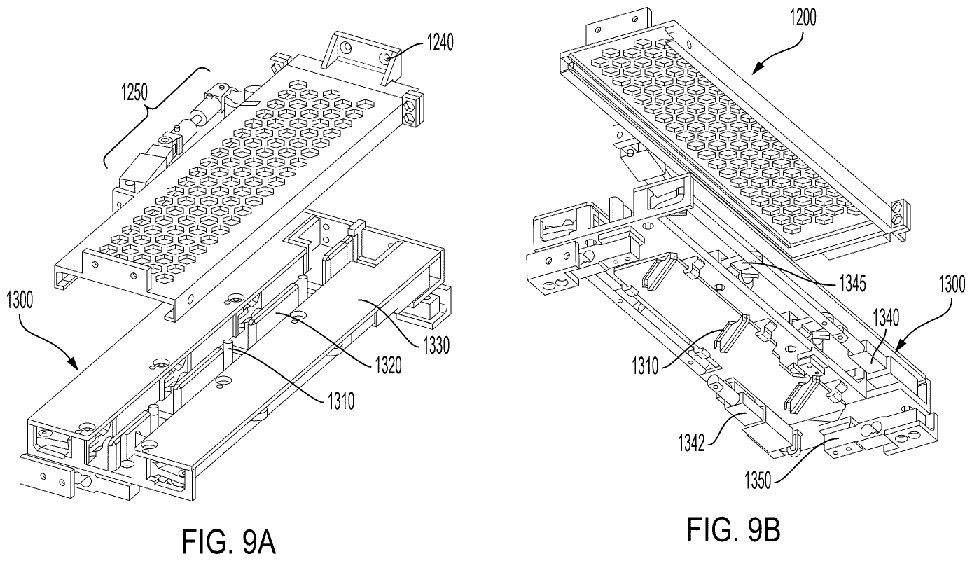

FIGS. 7A-B are schematic diagrams of a bioreactor system 1000 without (FIG. 7A) and with (FIG. 7B) bioreactor chamber nodes 1400 (FIG. 7B), in accordance with an embodiment of the invention. As used herein, a “bioreactor chamber node” is a single vessel bioreactor chamber, which can be used in the bioreactor systems taught herein. As shown in FIGS. 7A-B and 8A-B, the bioreactor chamber nodes 1400 can be used together in the bioreactor system 1000 (see FIG. 7B) and can be arranged into a set 1450 (see FIGS. 8A and 8B), or multiple such sets 1450, which can be installed into the bioreactor system 1000 (see FIG. 7B). A set of multiple bioreactor chamber nodes 1400 and associated components can be interconnected to allow for more efficient loading and unloading into an automated bioreactor system (such as bioreactor system 1000 of FIGS. 7A-B), thereby facilitating process scale-up. With reference to FIGS. 7A-B, the bioreactor system 1000 comprises a scaffold frame 1100 with one or more lateral supports 1130 that are configured to receive a chamber support assembly (see 1200 in FIGS. 9A and 9B, which receives support rails 1510 and 1520 of FIGS. 8A and 8B). The lateral supports 1130 (FIG. 7A) are configured to support one or more chamber support assemblies 1200, which in turn support sets 1450 of bioreactor chamber nodes 1400, such as in FIGS. 8A and 8B. With reference to FIG. 7B, a fluid manifold 1460 comprises a fill line fluid connection 1461 configured to permit filling of the sets 1450 of bioreactor chamber nodes, and a drain line fluid connection 1462 configured to permit draining of the sets 1450 bioreactor chamber nodes 1400. As used herein, it will be appreciated that references to filling can include filling bioreactor chamber nodes 1400 or other bioreactor chambers taught herein with biotic or abiotic materials for a bioreaction, including, for example, filling with cells and media for a bioreaction. References to draining can include draining a product of a bioreaction from bioreactor chamber nodes 1400 or other bioreactor chambers taught herein.

FIGS. 8A-B are schematic diagrams of a set 1450 of bioreactor chamber nodes 1400 (perspective view from above and below), in accordance with an embodiment of the invention. Here, the set 1450 of bioreactor chamber nodes is assembled into a bioreactor chamber assembly 1440, which includes the set 1450 of nodes 1400 and associated components (such as support rails 1510, 1520 and/or fluid manifold 1460) so that the nodes 1400 can be interconnected to be efficiently loaded into, and unloaded from, the automated bioreactor system. For this purpose, the bioreactor chamber assembly 1440 can include support rails 1510 and 1520, which can be received by the chamber support assembly 1200 (see FIGS. 9A-B and 13A-B) and which support multiple bioreactor chamber nodes 1400. Here (FIGS. 8A-B), the bioreactor chamber assembly 1440 further includes the fluid manifold 1460, which includes the fill line fluid connection 1461 and the drain line fluid connection 1462. The bioreactor chamber assembly 1440, with its included set 1450 of bioreactor chamber nodes 1400, can be loaded and unloaded as a unit within the bioreactor system 1000 (or other bioreactor systems taught herein). In an alternative arrangement, the fluid manifold 1460, fill line fluid connection 1461, and drain line fluid connection 1462 can be pre-installed on the scaffold frame 1100 rather than being a part of the bioreactor chamber assembly 1440.

With reference to FIGS. 7A-B and 9A-B, the bioreactor system 1000 further includes a chamber support assembly 1200, which mounts to the frame 1100 and is configured to receive a set 1450 of bioreactor nodes 1400. The bioreactor system 1000 also includes a chamber interface assembly 1300, which can include features for flow control, optical sensing, and weight measurement (FIGS. 9A-B).

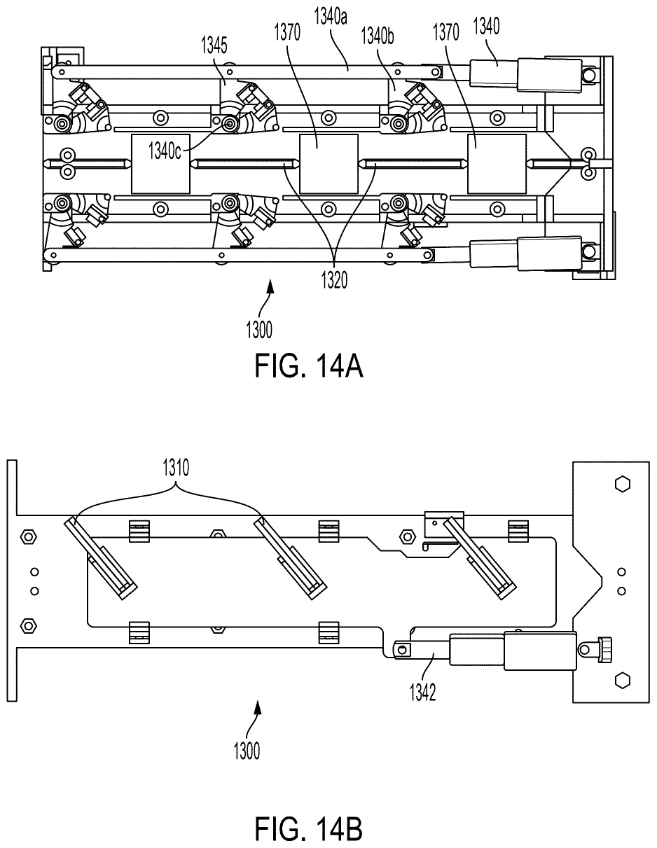

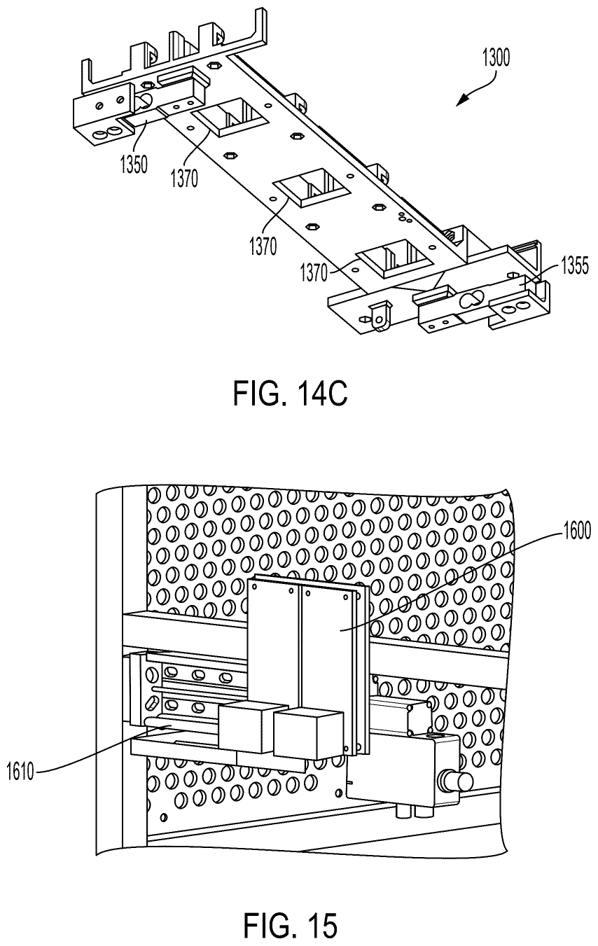

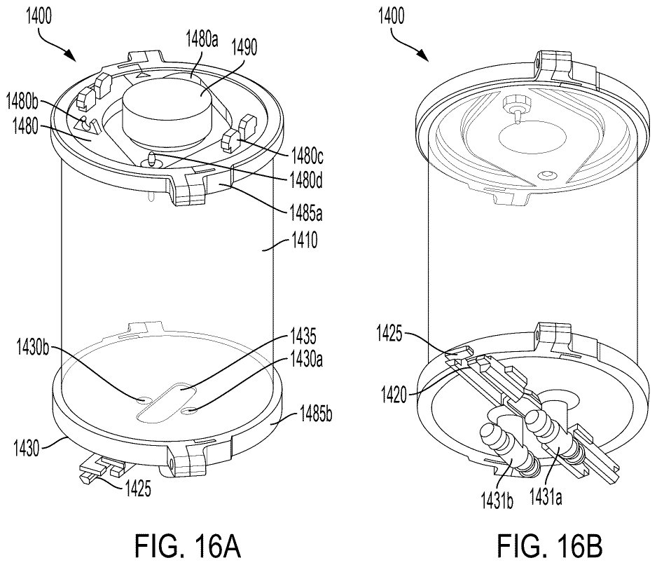

The chamber interface assembly 1300 includes an optical guide 1310 (FIGS. 9A-B) configured to aim an optical fiber at an optical access window 1435 (see FIGS. 16A-B) of a bioreactor chamber node 1400 (see FIGS. 7A-B) in the set 1450 of bioreactor chamber nodes 1400. Also shown in FIG. 9B are valve actuator 1340 and valves 1345 (described in connection with FIG. 14A), optical guide linear actuator 1342 (described in connection with FIG. 14B, and weight measurement module 1350 (described in connection with FIG. 14C).

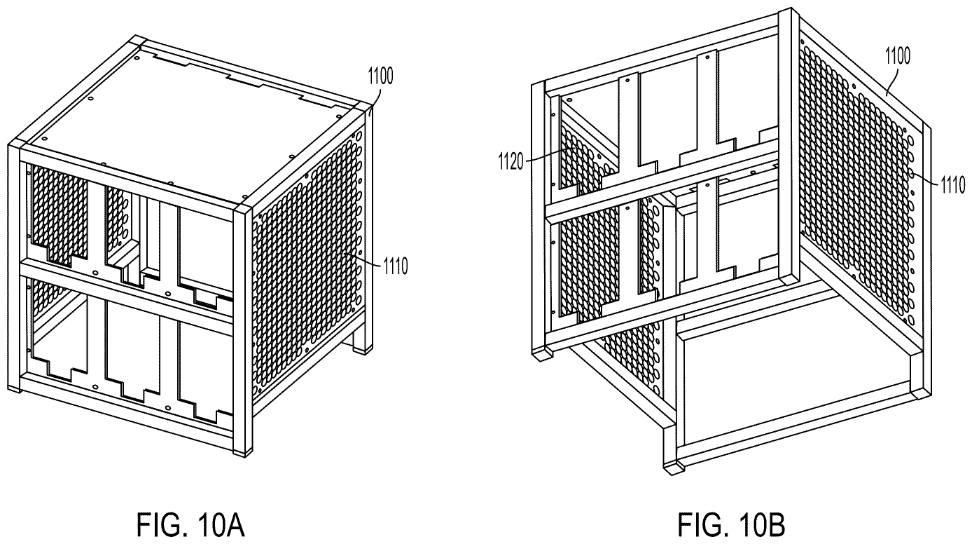

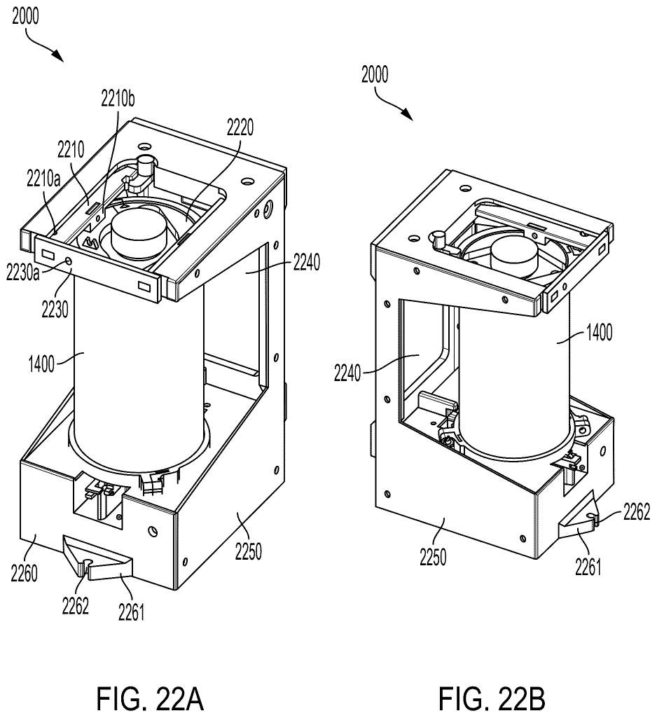

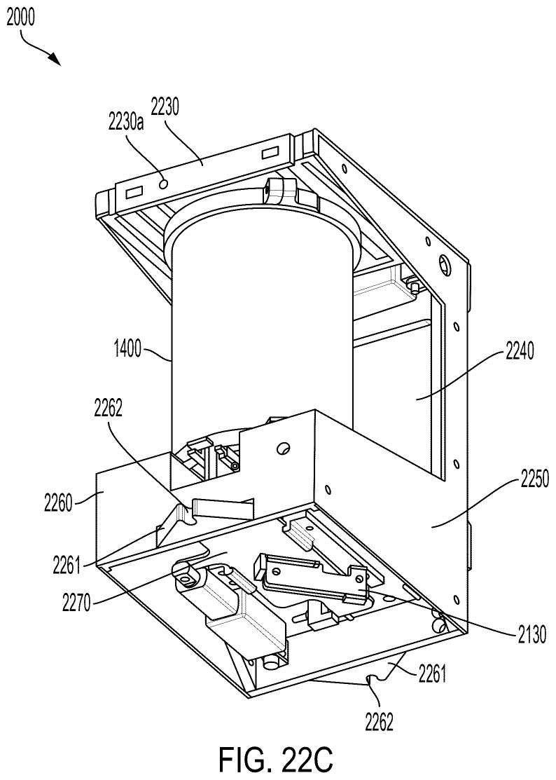

FIGS. 10A-B are schematic diagrams of the scaffold frame 1100 (perspective views from above and below), in accordance with an embodiment of the invention. The scaffold frame 1100 can include clearances 1120 for the bioreactor chamber assemblies (such as 1440 of FIGS. 8A-B), and can include an airflow panel, such as a perforated airflow panel 1110, within the scaffold frame 1100. The scaffold frame 1100 can be configured to mechanically mount the bioreactor system (such as system 1000 of FIGS. 7A-B) within an incubator (not shown in FIGS. 10A-B), for example by including features (for example, similar to feature reference number 2262, see FIGS. 22A-C and 23) that mechanically engage with a component of the incubator to permit agitation and/or mechanical stability of the bioreactor system within the incubator.

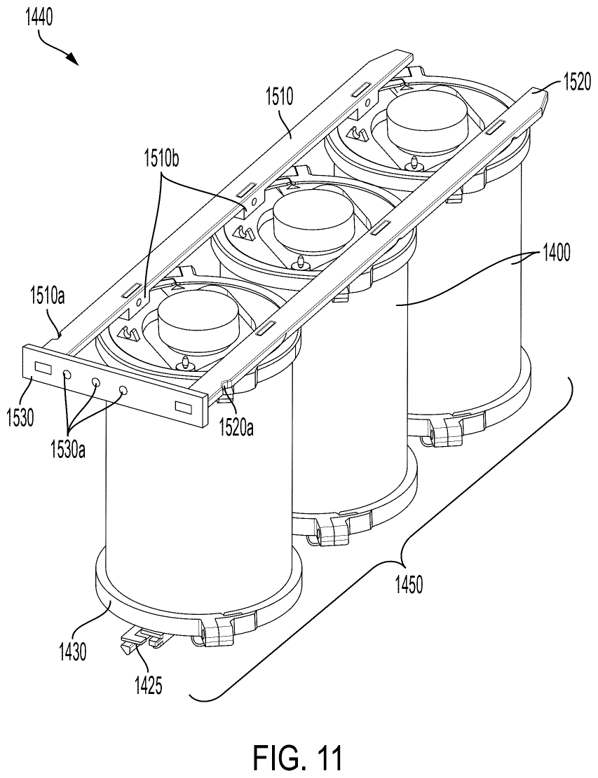

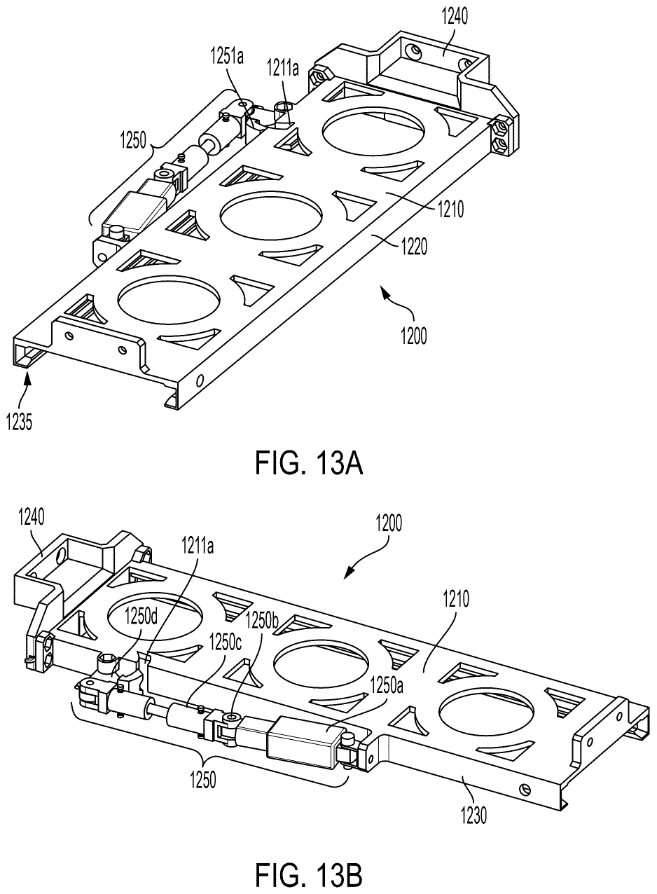

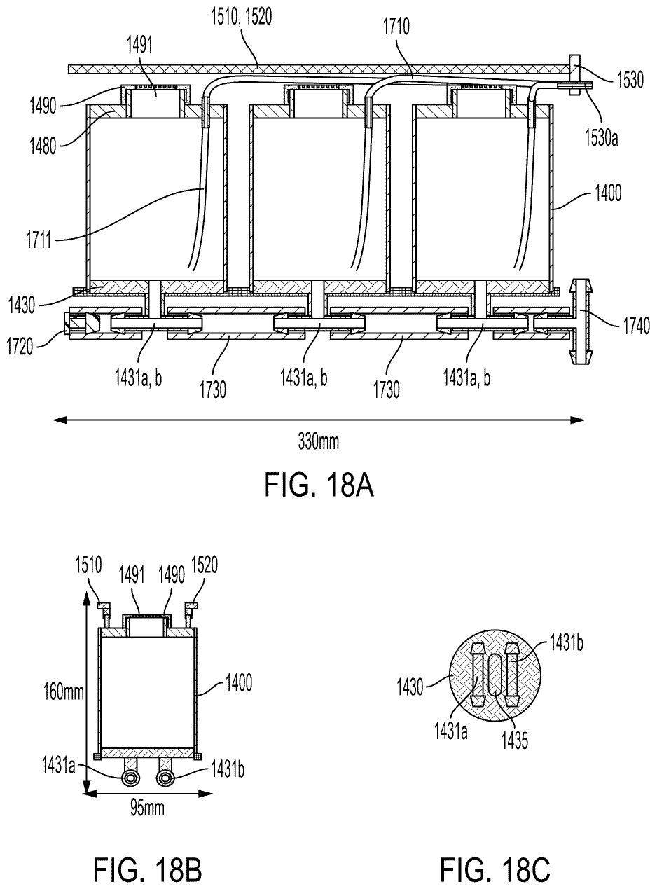

FIG. 11 is a schematic diagram of a bioreactor chamber assembly 1440, including a support rail 1510, 1520, in accordance with an embodiment of the invention. In FIG. 11, the support rail 1510, 1520 is configured to be slidingly mounted within the chamber support assembly 1200 (FIGS. 13A-B), for example by the support rail 1510, 1520 (see FIG. 11) being sized and shaped to slide within a corresponding opening 1235 (see FIG. 13A) in the chamber support assembly 1200 (see FIGS. 13A, 13B). The support rail 1510, 1520 (see FIG. 11) can include a bracket 1510b configured to mount a bioreactor chamber node 1400 of the set 1450. An engagement detent 1510a, 1520a of the support rail 1510, 1520 can be configured to engage with the chamber support assembly 1200 (see FIGS. 13A-B), for example to engage with a sprung-plunger within one or more of the support rail slide tracks 1220, 1230 (FIGS. 13A-B) of the chamber support assembly 1200. As shown in FIGS. 7A-B, the bioreactor system 1000 can include multiple sets 1450 of bioreactor chamber nodes 1400 (such as multiple bioreactor chamber assemblies 1440 of FIG. 11). For example, in FIGS. 7A-B, each level of the bioreactor system 1000 includes three sets 1450 (FIG. 7B) of bioreactor chamber nodes 1400. It will be appreciated that different numbers of levels and different numbers of sets 1450 per level can be used. With reference to FIG. 11, a support rail front plate 1530 can include one or more sample tubing ports 1530a. Sample tubing (not shown, see 1710 in FIG. 18A) can extend from the support rail front plate 1530 (FIG. 11), through each sample tubing port 1530a, to one or more of the bioreactor chamber nodes 1400. For example, there can be one sample tubing port 1530a for each bioreactor chamber node 1400 in the set 1450 (in FIG. 11, three sample tubing ports 1530a for each of the three bioreactor chamber nodes 1400 in the set 1450). A pair of parallel support rails 1510, 1520 can be configured to support the set 1450 of bioreactor chamber nodes 1400. The support rail front plate 1530 can include one or more sample tubing ports 1530a, with the support rail front plate 1530 being mounted across the pair of parallel support rails 1510, 1520. Although support rails 1510, 1520 are shown as being top support rails that support nodes 1400 from above, it will be appreciated that, in alternative embodiments, support rails 1510, 1520 can be on the side or bottom of the nodes 1400. The bioreactor chamber assembly 1440 can be used in the bioreactor systems taught herein and can be a consumable or disposable module that is used once and then disposed of. Alternatively, the bioreactor chamber assembly 1440 can be a reusable assembly.

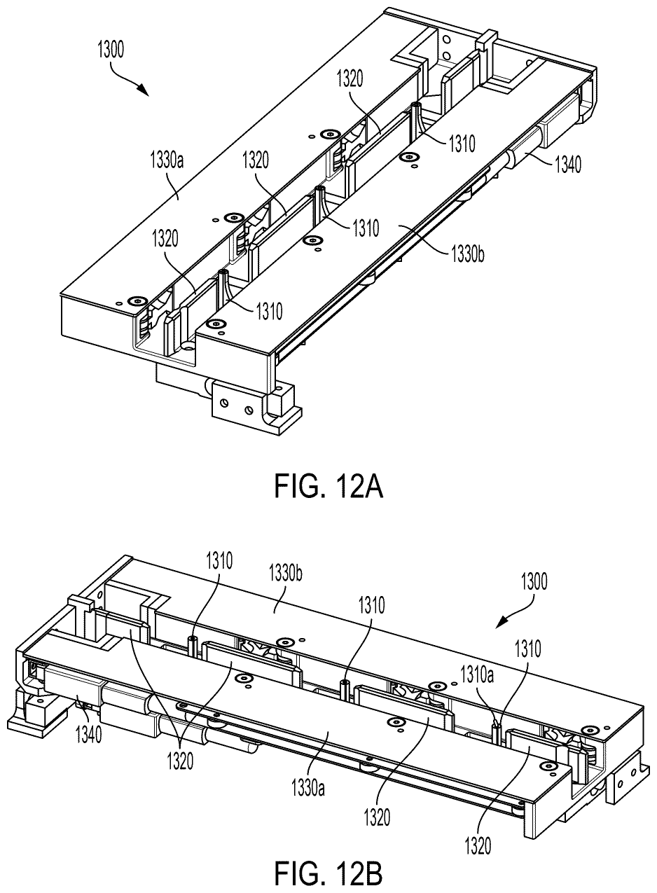

FIGS. 12A-B are schematic diagrams of the chamber interface assembly, and FIGS. 13A-B are schematic diagrams of the chamber support assembly, each in accordance with an embodiment of the invention. The chamber interface assembly 1300 is now discussed with reference to parallel items in FIGS. 9A-B and 12A-B; and the chamber support assembly 1200 is now discussed with reference to parallel items in FIGS. 9A-B and 13A-B. The chamber support assembly 1200 can include a fastening feature 1240 configured to fasten the chamber support assembly 1200 to the scaffold frame 1100 (FIGS. 7A-B) (for example, using bolts or screws). In addition, the chamber support assembly 1200 (FIG. 13A) can include an opening 1235 (FIG. 13A) that is configured to slidingly receive and mount the support rail 1510, 1520 (FIG. 11) of the bioreactor chamber assembly 1440. The opening 1235 can, for example, be an opening formed in a pair of support rail slide tracks 1220, 1230. A support assembly protection structure 1210 can serve to provide structural rigidity to the chamber support assembly 1200, and/or to provide mechanical protection for the bioreactor chamber nodes, and/or to prevent unintentional spills or leaks from affecting other components. Further, the chamber support assembly 1200 (FIGS. 9A, 9B, 13A, 13B) can also include a bioreactor chamber set lock 1250 configured to locate the bioreactor chamber assembly 1440 (FIG. 7B) having the set 1450 of bioreactor chamber nodes 1400 (FIG. 7B) in position after the bioreactor chamber assembly 1440 is inserted into the bioreactor system. The bioreactor chamber set lock 1250 inserts a latch 1251a into notch 1211a of the chamber support assembly 1200, thereby latching the bioreactor chamber set lock 1250 in place once the bioreactor chamber assembly 1440 is inserted into the bioreactor system. The bioreactor chamber set lock 1250 can include a linear actuator 1250a (FIG. 13B) attached by a pivot connection 1250b (FIG. 13B) to a lost motion element 1250c, which connects to a lever arm 1250d that actuates the latch 1251a to lock the bioreactor chamber assembly 1440 in position after the bioreactor chamber assembly is inserted into the chamber support assembly 1200. The linear actuator 1250a can be configured to extend to cause the engagement of the latch 1251a to lock the bioreactor chamber assembly 1440 into the chamber support assembly 1200, and the linear actuator 1250a can be configured to retract to cause the disengagement of the latch 1251a to permit removal of the bioreactor chamber assembly 1440 from the chamber support assembly 1200.

The chamber interface assembly 1300 (FIGS. 12A-B) can include a guide rail 1320 configured to fit with an alignment rail 1420 (FIG. 16B) of a bioreactor chamber node 1400 (FIGS. 18A, 18B) of the set. In addition, as shown in FIGS. 12A-B, the chamber interface assembly 1300 can include optical guides 1310 (shown here from the top side of the chamber interface assembly 1300) that can be configured to aim an optical fiber 1310a (FIG. 12B) at an optical access window of the bioreactor chamber nodes. The chamber interface assembly 1300 (FIGS. 9A, 9B, 12A, 12B) can also include a valve actuator 1340 configured to rotate valves 1345 (FIG. 14A) that are configured to close and open base tubing 1730 (see FIG. 18A) that is in fluid connection between the bioreactor chamber nodes 1400 (FIGS. 7A-B) of the set 1450 (FIG. 7B). Further, the chamber interface assembly 1300 (FIGS. 9A, 12A, 12B) can also include a cosmetic covers 1330a, 1330b.

FIGS. 14A-C are schematic diagrams showing the chamber interface assembly 1300, illustrating the operation of the valving, optical guides, and weight measurement module, in accordance with an embodiment of the invention. The valve actuator 1340 (FIG. 14A) can be configured to rotate multiple valves 1345 that are configured to close and open base tubing (not shown in FIG. 14A, see 1730 in FIG. 18A) through a pinch-close design. For example, the valve actuator 1340 can be configured to rotate all of the valves 1345 that are configured to close and open the base tubing for all of the bioreactor chamber nodes of one set of nodes. The valves 1345 can include a lost motion spring. The valves 1345 can, for example, be pinch valves, which can operate to open and close the base tubing. For example, the valve actuator 1340 can operate to extend and retract a valve actuator arm 1340a, which is attached to valve mechanism 1340b that is configured to rotate about pivot point 1340c. Upon extending and retracting of the valve actuator arm 1340b, the valve mechanism 1340b rotates about the pivot point 1340c to pinch closed, or remove a pinching to open, the base tubing (see tubing 1730 in FIG. 18A), such as by pinching the tubing against alignment rail 1420.

FIG. 14B is a schematic diagram showing a bottom view of the chamber interface assembly 1300, illustrating the operation of optical guides 1310, in accordance with an embodiment of the invention. The chamber interface assembly 1300 can, for example, include one optical guide 1310 for each bioreactor chamber node 1400 (FIGS. 7A-B) of a set 1450 (FIG. 7B), for example three optical guides 1310 (FIG. 14B) for a set of three bioreactor chamber nodes; or there can be more than one optical guide 1310 per node. An optical guide linear actuator 1342 can be configured to position the optical guide 1310 relative to the optical access window (not shown in FIG. 14B, see 1435 of FIG. 16A-B) of a bioreactor chamber node. For example, the linear actuator 1342 can extend and retract to move the optical guides to left and right. The optical guides 1310 can extend through an opposite side of the chamber interface assembly 1300 to be able to aim at the optical access window of a bioreactor chamber node (as shown by the top portions of 1310 extending through the top side of the chamber interface assembly 1300 in FIGS. 12A and 12B). In FIG. 14A, there are ports 1370 for optical access, through which optical guides 1310 can extend, or in some embodiments, through which optical guides 1310 can aim optical fibers (for example, through a transparent medium layer in ports 1370). (See also bottom view of ports 1370 in FIG. 14C). In one embodiment, there can be one optical fiber 1310a per bioreactor chamber node. In an alternative embodiment, the chamber interface assembly 1300 can include more than one optical fiber 1310a for each bioreactor chamber node 1400 (FIGS. 7A-B) of a set 1450 (FIG. 7B), for example by having two or more optical guides 1310 (FIG. 14B) per each chamber node 1400. For example, the system can perform more than one type of spectroscopy on a bioreactor chamber node 1400 through more than one optical fiber. For example, one optical fiber can be used to perform fluorescence spectroscopy on the bioreactor chamber node 1400, while another optical fiber can be used to perform Raman spectroscopy on the bioreactor chamber node 1400.

FIG. 14C is a schematic diagram showing a perspective view from below the chamber interface assembly 1300, illustrating the operation of a weight measurement module 1350, in accordance with an embodiment of the invention. The chamber interface assembly 1300 can, for example, include a weight measurement module 1350 that is configured to determine a weight of a bioreactor chamber node 1400, or of a set 1450 of bioreactor chamber nodes 1400 (FIG. 7B). The weight measurement module 1350 (FIG. 14C) can, for example, comprise a strain gauge 1355. The bioreactor chamber node 1400 (FIG. 11) can, for example, be configured to be suspended from the support rail 1510, 1520 (FIG. 11) in a mechanically compressed state, and the weight measurement module 1350 (FIG. 14C) can be configured to sense a weight added to the bioreactor chamber node via additional load on the base of the bioreactor chamber node when the weight is added to the bioreactor chamber node, which presses down on the strain gauge 1355 via the chamber interface assembly 1300.

FIG. 15 is a schematic diagram illustrating use of an optical multiplexer, in accordance with an embodiment of the invention. The bioreactor system can include an optical multiplexer 1600 configured to perform optical sensing of the bioreactor chamber nodes of the set. The optical multiplexer 1600 can be configured to restrict sensing to one bioreactor chamber node of the set at a given time; and can be further configured to change the one bioreactor chamber node to be sensed. For example, the optical multiplexer 1600 can include a linear stage 1610 to position an optical sensor relative to one or more optical fibers, such as by moving the optical multiplexer 1600 from one optical fiber to another. The optical fibers can be configured to perform optical sensing of the bioreactor chamber nodes, for example by being directed from the optical multiplexer 1600 to run through the optical guides 1310 (FIG. 14B), from which the optical fibers are aimed at the optical access windows 1435 (see FIGS. 16A-B) of the bioreactor chamber nodes.

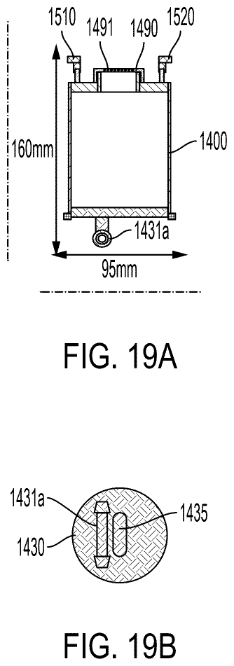

FIGS. 16A-B are schematic diagrams of a bioreactor chamber node 1400 (top view and bottom view), in accordance with an embodiment of the invention. The bioreactor chamber node 1400 can be used in the bioreactor systems taught herein, and can, for example, be a consumable or disposable module that is configured to be used once and then disposed of. Alternatively, the node 1400 can be a reusable module. The bioreactor chamber node 1400 can include a flexible, oxygen-permeable membrane 1410 configured to contain a biotic or abiotic material for a bioreaction. The flexible, oxygen-permeable membrane 1410 can, for example, be collapsible. The membrane 1410 can, for example, be or include one or more of silicone (and silicone derivatives such as PDMS), polypropylene, polystyrene, polytetrafluoroethylenes (PTFEs), and polyethylenes. The membrane 1410 can comprise a thickness of less than about 5 mil. The membrane 1410 can, for example, be polypropylene comprising a thickness of less than about 5 mil. The membrane 1410 can, for example, be sealed to the rigid top cap 1480 and sealed to the rigid bottom cap 1430; and it can, for example, comprise a volume of less than about 500 ml. A rigid top cap 1480 can be mounted to a top portion of the flexible, oxygen-permeable membrane 1410. The rigid top cap 1480 can be configured to mount to the support rail 1510, 1520 (see FIG. 11). For example, the rigid top cap 1480 (FIG. 16A-B) can be mounted to the support rail 1510, 1520 (of FIG. 11) by a portion of the rigid top cap 1480 being fitted into the bracket 1510b of the support rail 1510, 1520 (FIG. 11), or by using a clip 1480 c on the rigid top cap 1480 (FIG. 16A-B) to engage the bracket 1510b of the support rail 1510, 1520 (FIG. 11). The bioreactor chamber node 1400 (FIGS. 16A-B) can be configured to be suspended from the support rail 1510, 1520 (FIG. 11), for example by having the node 1400 (FIGS. 16A-B) hang from at least part of the rigid top cap 1480, or a clip 1480c (FIGS. 16A-B) of the rigid top cap, which can be held in the bracket 1510b of the support rail 1510, 1520 (FIG. 11) or by multiple such features in one or more of the support rails 1510, 1520 of FIG. 11. A rigid bottom cap 1430 (FIGS. 16A-B) can be mounted to a bottom portion of the flexible, oxygen-permeable membrane 1410, and the rigid bottom cap 1430 can include one or more fluid access ports 1430a, b (herein, an embodiment with one fluid access port 1430a, b is illustrated in FIGS. 19A-B, in addition to an embodiment with two fluid access ports 1430a, b in FIGS. 18B-C; other numbers of fluid access ports 1430a, b can be used). An optical access window 1435 can be configured to provide optical monitoring of the bioreaction by an optical fiber, for example by permitting light from an optical fiber to be transmitted through the optical access window 1435 and reflected back to permit sensing for monitoring the bioreaction). The bioreactor chamber node 1400 can be configured to be suspended from the support rail 1510, 1520 (see FIG. 11) in a mechanically compressed state. The one or more fluid access ports 1430a, b (FIGS. 16A-B) can connect to barbs 1431a, b. Typically, there are two fill ports 1430a, b and two barbs 1431a, b, one pair for filling and the other pair for dispensing. When there are two barbs 1431a, b, one of the barbs 1431a, b serves as a fill connection barb 1431a, in fluid connection with fill port 1430a, to provide fluid connection to base tubing 1730 (FIG. 18A), serving as fill base tubing. Another of the barbs 1431a, b serves as a dispense connection barb 1431b, in fluid connection with the dispense port 1430b, and is configured to provide fluid connection to base tubing 1730 (FIG. 18A) serving as dispense base tubing. Alternatively, a single fluid access port embodiment is described in FIGS. 19A-B, below, in which a single barb is used to connect to the base tubing, which serves as both fill base tubing and dispense base tubing. As shown in FIG. 16B, the bioreactor chamber node 1400 can include an alignment rail 1420 configured to align the node 1400 to a guide rail 1320 (see FIGS. 12A, 12B) of the chamber interface assembly 1300. The top cap 1480 (FIG. 16A) of the bioreactor chamber node 1400 can further include: a barb location 1480a for feed potential; a sample tubing clip 1480b; a clip 1480c configured to mount to the support rail 1510, 1520 (see FIG. 11); a sample tubing barb 1480d (FIG. 16A) configured to permit fluid connection with an interior of the flexible, oxygen-permeable membrane; a clamp ring 1485a (top) and 1485b (bottom); an interlock 1425; and a closure cap 1490 (such as a screw cap), which can, for example, be used for pipette access and/or venting. The rigid top cap 1480 can include an opening for the closure cap 1490. The interlock 1425 can, for example, be attached to the rigid bottom cap 1430, and is configured to permit connection between bioreactor chamber nodes 1400 without additional parts for that connection. In addition, the interlock 1425 can include a groove in its underside, which engages with the guide rail 1320 (see FIGS. 12A-B) to create alignment of the nodes 1400 with the optical guides 1310 (see FIGS. 12A-B). At the rear end of the guide rail 1320 (see FIGS. 12A-B), an end stop or end plate can prevent the nodes 1400 from moving too far or moving up off the surface of the guide rail. The top clamp ring 1485a (FIG. 16A) of the node 1400 can be configured to create a seal around a periphery of the rigid top cap 1480. The bottom clamp ring 1485b can be configured to create a seal around a periphery of the rigid bottom cap 1430. The rigid top cap 1480 and the rigid bottom cap 1430 can, for example, each be substantially circular in cross-section.

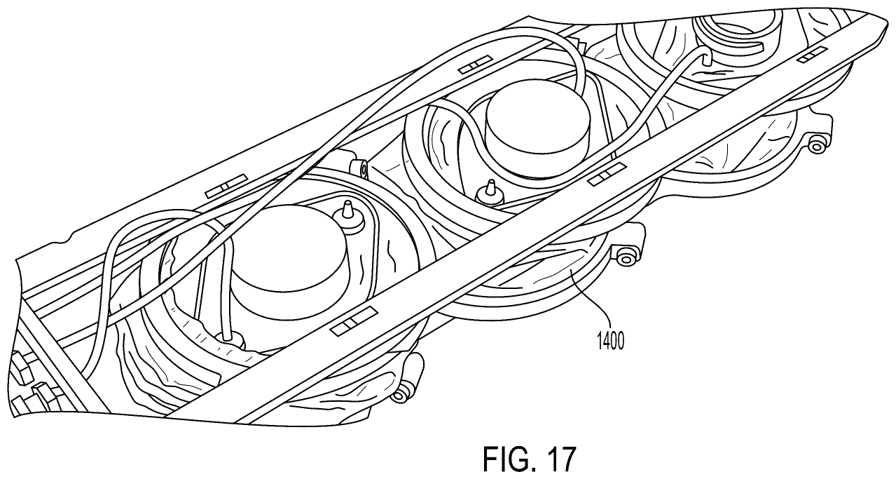

FIG. 17 is a schematic diagram showing bioreactor chamber nodes 1400 in a mechanically compressed state, in accordance with an embodiment of the invention. In FIG. 17, the mechanically compressed state is shown as one in which the bioreactor chamber nodes 1400 are considerably compressed and empty, which can be convenient, for example, for shipping of the component nodes or sets of nodes. In addition, once mounted to the bioreactor system and partially or fully filled, the bioreactor chamber nodes 1400 can be suspended in a state that is only partially or slightly mechanically compressed, between the chamber support assembly 1200 (FIGS. 12A and 12B) and chamber interface assembly 1300 (FIGS. 13A and 13B), to permit sensing of added or removed weight from the bioreactor chamber nodes 1400 by the weight measurement module 1350, as described in connection with FIGS. 14A-C (see also FIG. 7B, showing the nodes 1400 extending between the chamber support assembly 1200 and chamber interface assembly 1300).

FIGS. 18A-C are schematic diagrams illustrating connections to the bioreactor chamber nodes 1400, in accordance with an embodiment of the invention. As shown in FIG. 18A, the support rail front plate 1530 (which is attached to the support rail 1510, 1520) includes sample tubing ports 1530a, through which sample tubing 1710 extends to one or more of the bioreactor chamber nodes 1400. A dip tube 1711 can extend within the interior of the flexible, oxygen-permeable membrane 1410. Base tubing 1730 permits flow between the bioreactor chamber nodes 1400, for example via the fill connection barb 1431a or dispense connection barb 1431b. Here, it is noted that base tubing 1730 can serve as either fill base tubing or dispense base tubing, since a cross section is shown that can be through either a dispense side flow path or a fill side flow path, or that can be a cross section through a single flow path that is used for both dispensing and filling. It is also noted that the side view of FIG. 18A can show a cross section through either a fill connection barb 1431a or a dispense connection barb 1431b. Also shown are the rigid top cap 1480, rigid bottom cap 1430, and a closure cap 1490 that includes a vent 1491. FIG. 18B is a cross-section that shows both the fill connection barb 1431a and the dispense connection barb 1431b. In the bottom view of FIG. 18C, the optical access window 1435 is shown, which can, for example, be positioned on the rigid bottom cap 1430, such as between the two fluid connection barbs 1431a and 1431b. The optical access window 1435 can be or include a transparent region of the rigid bottom cap; which can comprise a region of sufficient optical clarity at sensing wavelengths (such as between 200 nm and 2000 nm) to enable direct optical interrogation of materials within the reaction chamber. For example, the optical access window 1435 can be a transparent region (such as transparent to sensing wavelengths, for example between 200 nm and 2000 nm) in an otherwise non-transparent region of the node 1400 (such as in the rigid bottom cap or rigid top cap). As another example, the optical access window 1435 can have different optical properties than the surrounding portions of the node, such as the flexible, oxygen-permeable membrane. Generally, the optical access window 1435 can be a flat material, such as a rigid flat material; and it can be a material that has reduced optical reflection off its surface, with transmission in the above-referenced sensing wavelengths, and with reduced fluorescence in the sensing wavelengths. Further, the optical access window 1435 and the optical guide 1310 (see FIG. 14B) can be configured such that the optical guide 1310 is configured to aim the optical fiber at the optical access window 1435 with a perpendicular angle of incidence to the surface of the optical access window 1435, such as within plus or minus 5 degrees from a perpendicular angle of incidence. The optical guide linear actuator 1342 (FIG. 14B) can, for example, be configured to maintain such an angle of incidence by moving the optical guide 1310 relative to the optical access window 1435. Generally, the flexible, oxygen-permeable membrane 1410 is not a suitable optical interrogation because it is insufficiently rigid and flat, and thus the angle of incidence is variable and unpredictable. The transparent region of the optical access window 1435 (FIG. 18C) can, for example, comprise at least one of: acrylic, glass, quartz, polystyrene, polycarbonate, sapphire, polypropylene and polyethylene. For example, the transparent region can be or include acrylic or glass. The optical access window 1435 can be an elongated transparent region of the rigid bottom cap 1430. The elongated transparent region of the optical access window 1435 can, for example, extend substantially parallel to the alignment rail 1420 (FIGS. 16A-B). In addition, the fill connection barb 1431a and the dispense connection barb 1431b can be elongated fluidic barbs extending substantially parallel to the alignment rail 1420 (FIGS. 16A-B).

A fluid line end stop 1720 (FIG. 18A) can be a fill line end stop or a drain line end stop (depending on whether the end stop is used in the fill fluid path or the dispense fluid path). The valves 1345 (FIG. 14A) can be configured to isolate each of the plurality of bioreactor chamber nodes 1400 from each other during incubation of a bioreaction, for example by pinching closed the base tubing 1730 between the bioreactor chamber nodes 1400. A fluid end fitting 1740 can provide fluid connections to the fluid manifold 1460, including the fill line fluid connection 1461 (FIGS. 8A-B). The fill line fluid connection 1461 can be configured to permit filling of all the bioreactor chamber nodes 1400 of the set in a single filling, for example by including fluid access ports of the bioreactor chamber nodes 1400 in a set 1450 (of FIG. 11) in a flow series relationship, with the valves 1345 (FIG. 14A) open on the base tubing 1730 between the nodes 1400 in the set. The fill line fluid connection 1461 (FIG. 8) can be configured to permit filling of all of the plurality of bioreactor chamber nodes 1400 (FIG. 7B) of the bioreactor system 1000 (FIGS. 7A-B) in a single filling, for example by having a flow path from a fill line fluid connection 1461 to fluid access ports of all sets 1450 of nodes 1400 on all levels of a bioreactor system 1000 (FIGS. 7A-B).

FIGS. 19A-B are schematic diagrams showing an embodiment of the bioreactor chamber node 1400 that includes a single fluid access port, in accordance with an embodiment of the invention. In this embodiment, a single fluid connection barb 1431a is used, which connects to a single fluid access port 1430a (FIGS. 16A-B), for use in both filling and dispensing. In FIG. 19B, the single fluid connection barb 1431a is shown from a bottom view, positioned next to the optical access window 1435 in the rigid bottom cap 1430.

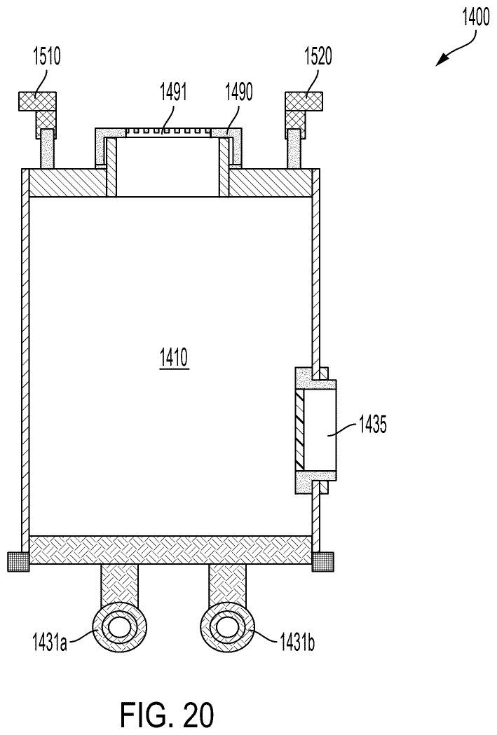

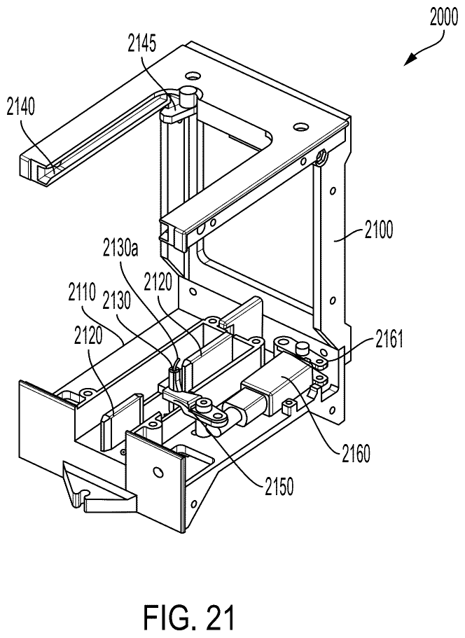

FIG. 20 is a schematic diagram showing an embodiment of a bioreactor chamber node 1400 in which the optical access window 1435 is mounted to the flexible, oxygen-permeable membrane 1410, for example in a position where it can be optically interrogated from the side of the bioreactor chamber node 1400, in accordance with an embodiment of the invention. Also depicted in FIG. 20 are the support rail 1510, 1520, closure cap 1490, vent 1491, flexible, oxygen-permeable membrane 1410, and barbs 1431a, b.

FIG. 21 is a schematic diagram of a bioreactor system 2000 for a single bioreactor chamber node 1400 (see FIGS. 22A-C), in accordance with an embodiment of the invention. The bioreactor system 2000 (FIG. 21) includes a mounting frame 2100 configured to support a single bioreactor chamber node 1400 (FIGS. 22A-C). The mounting frame 2100 (FIG. 21) is configured to receive a support rail 2210, 2220 (see FIGS. 22A-C) that is configured to mount to the single bioreactor chamber node 1400 (FIGS. 22A-C). For example, the mounting frame 2100 (FIG. 21) can include a sprung-plunger detent 2140 for positive engagement when loading. A bottom chamber mount 2110 comprises a guide rail 2120 configured to fit with an alignment rail (not shown) of the single bioreactor chamber node 1400 (FIGS. 22A-C). The bioreactor system 2000 (FIG. 21) further includes a lock mechanism for the single bioreactor chamber node 1400 (FIGS. 22A-C). The lock mechanism (FIG. 21) can, for example, include an upper locking arm 2145 to constrain the rigid top cap of the single bioreactor chamber node without interfering with the fluid connectors; a lower locking arm 2150 to constrain the rigid bottom cap of the single bioreactor chamber node without interfering with the fluid connectors; an actuator 2160 of the lower locking arm 2150, and a lost motion element 2161 on the actuator 2160 to prevent damage to the fluidic connectors. An optical guide 2130 is configured to aim an optical fiber 2130a at an optical access window (not shown) of the single bioreactor chamber node.

FIGS. 22A-C are schematic diagrams of the bioreactor system of FIG. 21, showing a single bioreactor chamber node 1400 installed in the system, in accordance with an embodiment of the invention. Here, the bioreactor system 2000 includes the support rails 2210, 2220, which are slidingly engaged with the mounting frame 2100. The support rails 2210, 2220 can include one or more brackets 2210b (on one rail or both rails), configured to mount to the single bioreactor chamber node 1400, for example by receiving a top cap of the node 1400. In addition, the support rails 2210, 2220 can include one or more engagement features 2210a (on one rail or both rails), configured to mount to the mounting frame 2100. For example, the engagement feature 2210a can be a detent element (e.g., a mechanical or magnetic detent) configured to engage with the sprung-plunger 2140 in the mounting frame 2100. A support rail front plate 2230 can include at least one sample tubing port 2230a. A pair of parallel support rails 2210, 2220 can be configured to support the single bioreactor chamber node 1400. The support rail front plate 2230 can include one or more sample tubing ports 2230a, with the support rail front plate 2230 being mounted across the pair of parallel support rails 2210, 2220. The mounting frame 2100 (FIG. 21) can include one or more open sides 2240 (FIGS. 22A-B) to permit air flow around the single bioreactor chamber node 1400, and can include a side panel 2250 and front panel 2260. In an analogous fashion to the bioreactor chamber nodes 1400 of FIGS. 16A-B, and 18A-C (with similar components not shown here), the single bioreactor chamber node can include a flexible, oxygen-permeable membrane configured to contain a biotic or abiotic material for a bioreaction. The bioreactor system of FIG. 21, 22A-C, and 23 with single bioreactor chamber node 1400 can be configured to permit testing of a bioreaction in a single bioreactor chamber before performing in multiple chambers and/or at a larger scale. The same type of bioreactor chamber node 1400 as taught elsewhere herein can be used.

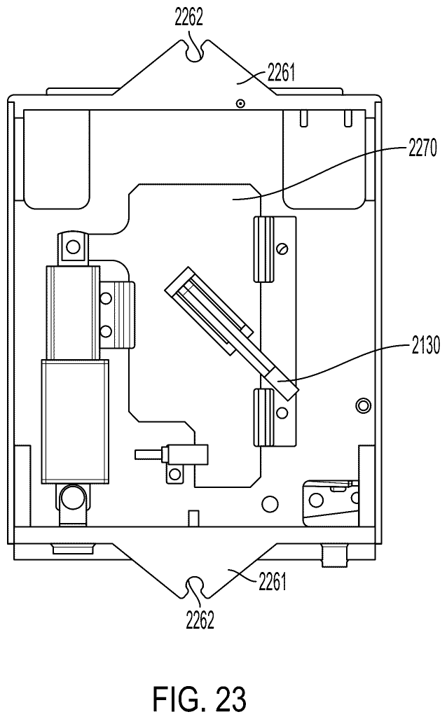

FIG. 23 is a schematic diagram showing a bottom view of the bioreactor system of FIGS. 21 and 22A-C, in accordance with an embodiment of the invention. The mounting frame 2100 (FIG. 21) can be configured to mechanically mount the bioreactor system within an incubator, for example by including bracket 2261 (FIG. 23) with engagement notch 2262 to allow rigid fixing to the incubator shaker plate. An optical slide element 2270 can be configured to move the optical guide 2130 (shown here from below) relative to the optical access window of the single bioreactor chamber node.

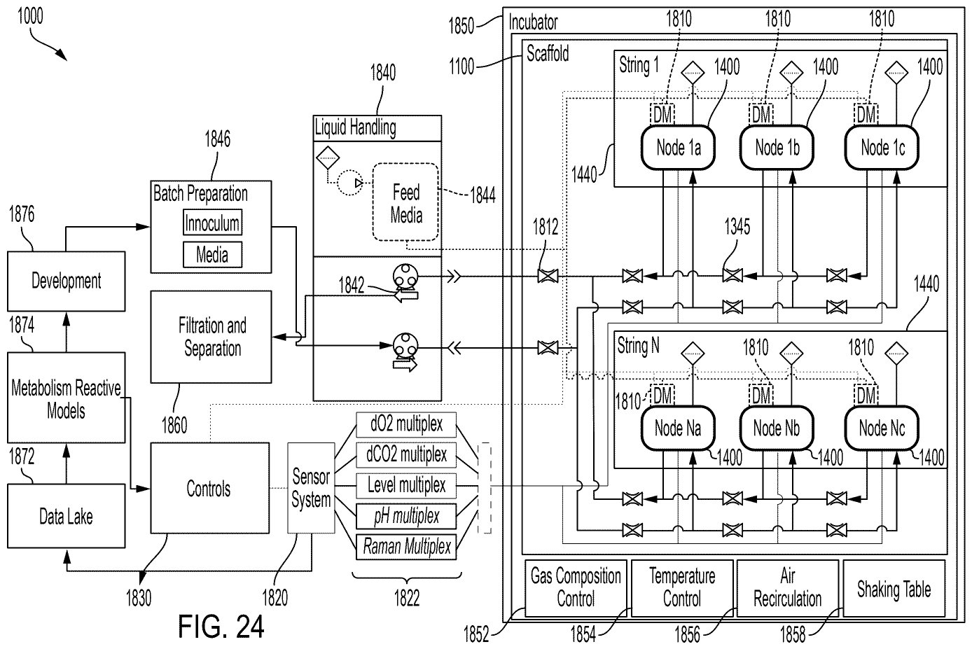

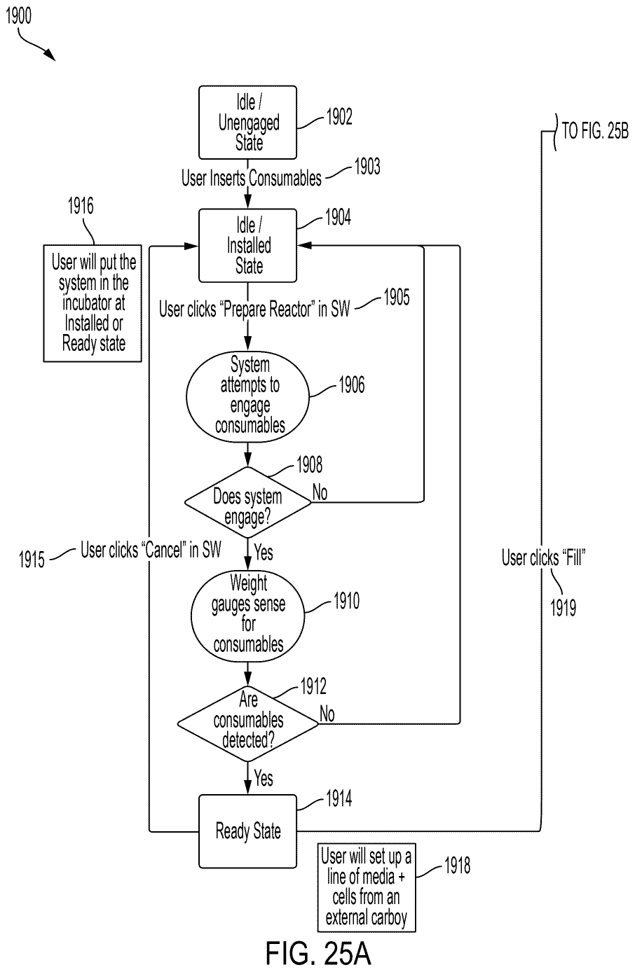

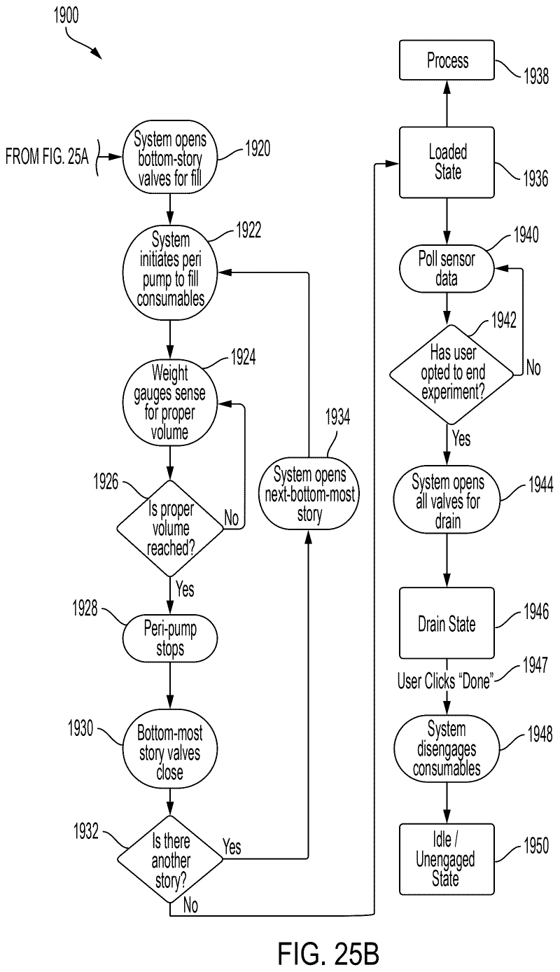

In another embodiment, a method of performing a bioreaction includes: a) filling a set of bioreactor chamber nodes arranged in a bioreactor system, the filling of the set of bioreactor chamber nodes being performed in a single filling step; b) isolating the set of bioreactor chamber nodes when each bioreactor chamber node is filled; c) performing a bioreaction within the isolated set of bioreactor chamber nodes so filled; and d) opening at least one valve to drain a product of the bioreaction in the set of bioreactor chamber nodes. The bioreactor system employed in the method can include any of the bioreactor systems taught herein. The method can include filling of all of the plurality of bioreactor chamber nodes of the bioreactor system in a single filling. Further, the method can include use of a controlled environment containment taught herein, surrounding the bioreactor system, including with gas surrounding the system. Further, the method can include performance of any of the methods taught in connection with FIGS. 24 and 25A-B herein.

In another embodiment, a method of scaling biomanufacturing comprises: a) performing a bioreaction in a bioreactor chamber node comprising a volume of between about 50 mL and about 50 L; and b) subsequently culturing cells in a plurality of bioreactor chamber nodes each comprising a volume of between about 50 mL and about 50 L arranged in a bioreactor system taught herein, comprising the plurality of bioreactor chamber nodes each comprising the volume of between about 50 mL and about 50 L in the bioreactor system taught herein.

In another embodiment, a bioreactor system comprises a plurality of bioreactor chamber nodes arranged in a bioreactor system, the plurality of bioreactor chamber nodes being configured to be filled in a single filling step. The plurality of bioreactor chamber nodes are configured to be isolated when at least each bioreactor chamber node of the single filling step is filled, and to perform a bioreaction within the isolated plurality of bioreactor chamber nodes so filled. At least one valve is configured to be opened to drain a product of the bioreaction in the plurality of bioreactor chamber nodes. A controlled environment containment can surround the bioreactor system. A volume between the bioreactor system and the controlled environment containment may comprise a gas. The controlled environment containment may comprise an incubator.

FIG. 24 is a system architecture diagram for a bioreactor system, in accordance with an embodiment of the invention. The system 1000 includes consumable components such as bioreactor chamber nodes 1400; dose management components 1810; the bioreactor chamber assemblies 1440 (which include the chamber nodes 1400); and a system fluid manifold 1812. The bioreactor chamber nodes 1400 can be the replicable unit that maintains conditions for the biology involved in the bioreaction, identically (or substantially identically) between any scale. The node 1400 provides easy diffusion for gas and heat through its boundary and can, for example, isolate a volume equivalent to a shake flask. The dose management components 1810 can, for example, allow for continuous feed of each node 1400 independently. The bioreactor chamber assemblies 1440 include a set of nodes 1400 that can be inserted as a single physical assembly. The system fluid manifold 1812 serves as a fluidic connector that combines the fluidics connections of the bioreactor chamber assemblies 1440 into a single fluid path to enter and exit the incubator 1850 (e.g., a single fill path and a single drain path for the bioreactor system 1000). For example, the system fluid manifold 1812 can distribute a single fluidic fill path to a plurality of sets of the bioreactor chamber nodes 1400; and a system fluid manifold 1812 can collect (aggregate) a plurality of sets of the bioreactor chamber nodes 1400 into a single fluidic drain path.

Further, the system 1000 of FIG. 24 includes durable components such as the scaffold frame 1100; a sensor system 1820; controls 1830; and liquid handling module 1840. The scaffold frame 1100 supports bioreactor chamber assemblies 1440 within the incubator 1850; provides mechanical fixing to the incubator 1850; and aligns the bioreactor chamber assemblies 1440 with valves, optical sensors, and other sensors. The valves 1345 can, for example, be pinch valves to ensure no breaking of fluidic paths. The sensor system 1820 can, for example, receive an optical signal from an optical fiber. Based on input from the optical fiber, the sensor system 1820 can be configured to measure one or more of dO₂ (dissolved oxygen), dCO₂ (dissolved carbon dioxide), pH, and level in the bioreactor chamber nodes, and can perform Raman spectroscopy. The sensor system 1820 can, for example, use optical fibers 1310a in optical guides 1310 (see FIG. 12B) to measure the quantities 1822. The quantities 1822 can, for example, be multiplexed using an optical switch (or optical multiplexer, such as optical multiplexer 1600 of FIG. 15). The controls 1830 (FIG. 24) can, for example, use the data collected from the optical measurements to be fed back into the monitoring of the bioreaction.

The liquid handling module 1840 of FIG. 24 can, for example, implement pumping of fluids by peristaltic pumps 1842 (including for filling and draining), to prevent contamination and to enable rapid changeover. Feed media 1844 (when applicable) can be collected in a tank and fed on demand to each node 1400. Batch preparation 1846 can include innoculum and media, which can be fed into the liquid handling module 1840.

In addition, the system 1000 of FIG. 24 includes systems that may be pre-existing systems used in other contexts. For example, incubator 1850 can be a type of incubator that is used for conventional flasks in other contexts. The incubator 1850 can, for example, include gas composition control 1852, which can be used for the whole chamber to enable uniform distribution of O₂, CO₂, or other gases. Further, the incubator 1850 can, for example, include temperature control 1854, so that the air temperature within the chamber is controlled to the same degree as a conventional flask incubator. Further, the incubator 1850 can include air recirculation 1856, since the scaffold frame 1100 and nodes 1400 may provide greater air resistance than typical contents of an incubator, and hence additional airflow may be required to ensure homogenization of the system. In addition, the incubator 1850 can include a shaking table 1858, which motivates the scaffold frame 100 and bioreactor system to ensure uniform agitation. Filtration and separation systems 1860 can, for example, be standard filtration and separation systems that can be used after the bioreaction is complete.

Although an incubator 1850 is provided in embodiments taught herein, it will be appreciated that an incubator is one example of a controlled environment containment that can surround the bioreactor systems taught herein. A controlled environment containment (for example, an incubator or other gas-based or air-based controlled environment container) can be used to surround any of the bioreactor systems taught herein. A volume between the bioreactor system and the controlled environment containment can comprise a gas, such as air. The controlled environment containment can comprise an incubator. An incubator can be a module that includes gas concentration control, thermal environment control, and a mechanical shaker control.

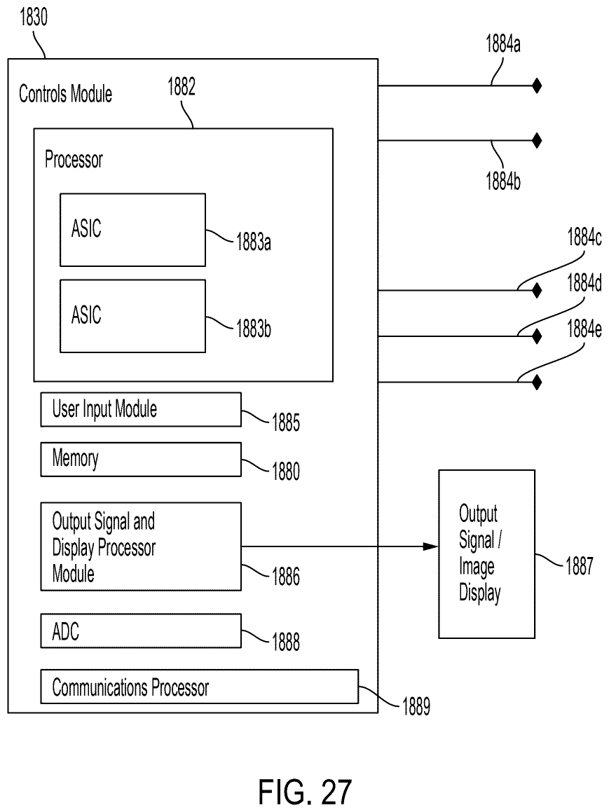

The system 1000 of FIG. 24 can also include data management components, such as data lake 1872, in which data that is gathered can be collected to monitor the effects of different growth conditions and the behavior of drugs across the system; metabolism reactive models 1874 (or other models 1874); and development components 1876, with which data collected during operations can be fed back to developers for iteration purposes. For example, the sensor system 1820 can be configured to provide data that is the result of its measuring of the quantities 1822 to the data lake 1872, which can be provided as input to the models 1874. The controls 1830 can be configured to analyze the data that is the result of the measuring of the quantities 1822 (such as based on output of the models 1874) and to use the analysis of such quantities 1822 to further control one or more of: the measuring by the sensor system 1820, operation of the sensor system 1820, operation of the optical guides, and operation of the optical multiplexer; operation of the valves; operation of the weight measurement system; the dose management components 1810; the operation of the system fluid lines 1812; the operation of the pumps 1842; the operation of the liquid handling module 1840; the control or input to development components 1876; and the batch preparation 1846. Such functions of the controls module 1830 can be performed using software and/or hardware controllers described below in connection with FIG. 27.

FIGS. 25A-25B are a process flow diagram 1900 for use of a bioreactor system, in accordance with an embodiment of the invention. These processes can likewise be implemented, for example, using software and/or hardware controllers operated by controls module 1830, as described further below in connection with FIG. 27. In FIG. 25A, the process begins in an idle/unengaged state 1902, at which the user inserts 1903 consumables such as bioreactor chamber assembles 1440 with nodes 1400. The process then enters an idle/installed state 1904. The user then clicks “Prepare Reactor” 1905 in a software interface, upon which the system attempts 1906 to engage consumables. At 1908, a decision is made whether or not the system has engaged. If not, the flow returns to the idle/installed state 1904. If yes, the weight measurement components sense 1910 for consumables (such as nodes 1400). At 1912, a decision is made whether or not consumables are detected. If not, the flow returns to the idle/installed state 1904. If yes, the system enters a ready state 1914. If the user then clicks “Cancel” 1915 in a software interface, the system returns to the idle/installed state 1904. As indicated at 1916, the user will put the system into the incubator at either the idle/installed state 1904 or the ready state 1914. As indicated at 1918, the user will set up a line of media and cells from an external carboy. Next, the user clicks “Fill” 1919 on a software interface.

The process continues in FIG. 25B, in which the system opens 1920 the bottom-story valves for fill. The system then initiates 1922 the peristaltic pump to fill the consumables. The weight gauges then sense 1924 for the proper volume in the consumables (such as nodes 1400). A decision 1926 is then made whether or not the proper volume is reached. If no, the weight gauges sense again at 1924. If yes, the peristaltic pump stops 1928. The bottom-most story valves then close 1930. At 1932, a decision is made whether or not there is another story of bioreactor chamber nodes 1400 to fill. If yes, the system opens 1934 the next-bottom-most story, and the process returns to 1922 to initiate the peristaltic pump to fill the consumables. If no (that is, there is not another story), the process moves to the loaded state 1936. In this state, the process 1938 of the bioreactor system occurs while the chambers are isolated and the bioreaction is incubated in each isolated chamber. While that is occurring, the system polls 1940 the sensor data. At 1942, a decision is made whether or not the user has opted to end the experiment. If no, the sensor is polled again at 1942. If yes, the system opens 1944 all valves for draining the system. The system then enters a drain state 1946. The user then clicks “Done” 1947 on a software interface. The system then disengages 1948 the consumables (nodes 1400). The system then enters an idle/unengaged state 1950.

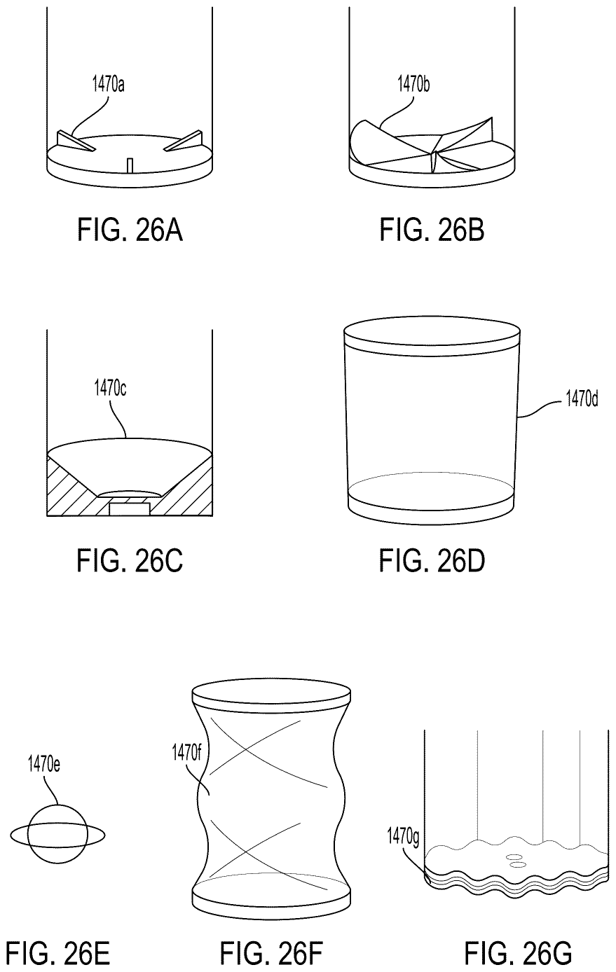

FIGS. 26A-G are schematic diagrams illustrating features for mixing in bioreactor chamber nodes, in accordance with an embodiment of the invention. These illustrate inclusion of a feature 1470a-g configured to promote mixing in the bioreactor chamber node. Mixing can assist to homogenize nutrient and gas composition throughout the fluid. On an orbital shaking table, the flows introduced are mainly radial (away from the center) and tangential (around the circumference). This creates challenges as the liquid may rotate in a constant configuration without significant mixing. By inducing some axial flow (along the axis of gyration and along the axis of the vessel), convection like currents are created, resulting in better mixing. In conventional bioreactors this is done through an impeller within the flask, but as well as introducing complexity to the vessel itself, this creates shear for the cells and can hinder growth. In shake flasks, this mixing is effected through walls angled in towards the center of the container and a shallow fill level. This is highly inefficient with use of space inside the incubator and therefore limits the total volume of liquid that can be run within a single cycle.

In accordance with an embodiment of the invention, static features can be included in the bioreactor chamber nodes so that the movement of fluid relative to these features, caused for example by an orbital shaker, can induce flow patterns to promote mixing. As shown in FIG. 26A, baffles 1470a can be included in the rigid bottom cap of the bioreactor chamber node. Baffles 1470a, such as vertical baffles, impede tangential flow within the container, forcing it to move upwards along the axis of the vessel and creating eddies on the rear side. As shown in FIG. 26B, tangential ramps 1470b can be included in the rigid bottom cap of the bioreactor chamber node. Smooth ramp transitions direct tangential flows up along the axis of the container while reducing stagnant regions within the vessel where cells might collect. The peak of the ramp can utilize a sharp angle change to effect separation of the flow up the ramp, to prevent the flow from moving back down the other side of the ramp. As shown in FIG. 26C, radial ramps 1470c can be included in the rigid bottom cap of the bioreactor chamber node. For example, an upwards slope can start far from the edge of the container and raise towards the edge, thereby diverting radially. As shown in FIG. 26D, there can be a cross section transition 1470d between the rigid bottom cap and the rigid top cap of the bioreactor chamber node. For example, by transitioning from a fully circular to an oval cross section between the bottom and the top of the flask, or by having ovals which are rotated 1470e (see FIG. 26E) through as much as 90 degrees between the bottom and the top, a continuously changing cross section will cause a tangential flow to turn into a radial or axial flow. As shown in FIG. 26F, the bioreactor chamber node can include a membrane twist 1470f, for example through rotation of the rigid top cap relative to the rigid bottom cap of the bioreactor chamber node. By twisting the top of the membrane relative to the bottom of the membrane, around its own axis, a helix pattern is created in the membrane, creating corrugations in the cross section which will turn tangential flows into axial and radial flows. As shown in FIG. 26G, a corrugated membrane 1470g can be included in the bioreactor chamber node. For example, features 1470g in the top and base of the vessel can create a corrugated membrane around its circumference, although the nature of the membrane may mean that these corrugations will exist only close to the region where the membrane is secured. A small distance from the rigid features, hydrostatic pressure will cause the membrane to form a cylinder. The vessel will therefore transition between corrugated regions and cylindrical regions. This will convert tangential flow to radial flow in the region of corrugation and radial flow to axial flow in the transition from the corrugated region to the cylindrical region. It will be appreciated that other features can be included to promote mixing in the bioreactor chamber nodes.

FIG. 27 is a schematic block diagram of a controls module 1830 (of FIG. 24) in a bioreactor system in accordance with an embodiment of the invention. The bioreactor system can include the controls module 1830 (FIG. 27), which includes at least one memory 1880, and at least one processor 1882, coupled to the at least one memory 1880. The processor 1882 is individually or collectively configured to, in a manner automated by the at least one processor 1882, perform a variety of functions provided herein, such as: measuring by the sensor system 1820 (FIG. 24), operation of the sensor system 1820, operation of the optical guides, and operation of the optical multiplexer; operation of the valves; operation of the weight measurement system; the dose management components 1810 (FIG. 24); the operation of the system fluid lines 1812 (FIG. 24); the operation of the pumps 1842 (FIG. 24); the operation of the liquid handling module 1840 (FIG. 24); the control or input to development components 1876 (FIG. 24); and the batch preparation 1846 (FIG. 24); and the flow process of FIGS. 25A-B.

Various techniques set forth herein are implemented using a controls module 1830 (FIG. 27), and can include computer implemented components, such as those of processor 1882. Control and processing techniques discussed herein can be implemented using software or hardware, or a mixture of both. For example, controls module 1830 can include one or more processors 1882, which can for example include one or more Application Specific Integrated Circuits (ASICs) 1883a, 1883b; application software running on one or more processors 1882 of the controls module 1830; and sensor and/or actuator lines 1884a-e delivering electronic signals to and from processors, memories, electronics components, and controllers, in which the signals can deliver electronic signals to and from any actuated components within systems set forth herein (including linear actuators). The controls module 1830 can also include user input module 1885, which can include components (such as a keyboard, touch pad, and associated electronics in connection with the processor 1882 and memory 1880) to receive user input such as the input described in connection with FIGS. 25A-B. The controls module 1830 can also include a memory 1880 to store information, and to implement procedures under control of computer hardware and software. Further, the controls module 1830 can include an Output Signal and Display Processor module 1886, which can generate an output signal and/or image display 1887, such as for use by the user of the flow process described in FIGS. 25A-B. An Analog-to-Digital Converter 1888 can be used by the controls module 1830 as needed to convert analog to digital signals. It will be appreciated that other control hardware may be used.

Portions of the above-described methods and systems can be implemented using one or more computer systems, for example to permit operation and control of a bioreactor system. For example, techniques can be implemented using hardware, software, or a combination thereof. When implemented in software, the software code can be executed on any suitable processor or collection of processors, whether provided in a single computer or distributed among multiple computers. For example, the processor 1882 can be provided in a single component, or distributed amongst multiple components, and can communicate over a communications network via communications processor 1889, including to permit control of the bioreactor system over a communications network.

### REFERENCES

Barcelos MC, Lupki FB, Campolina GA, Nelson DL, Molina G. The colors of biotechnology: general overview and developments of white, green and blue areas. FEMS microbiology letters. 2018 November; 365(21): fny 239.
El-Chichakli B, von Braun J, Lang C, Barben D, Philp J. Policy: Five cornerstones of a global bioeconomy. Nature. 2016 Jul. 14; 535(7611):221-3.
Kähler F., Carus M., Porc O., Vom Berg C. Turning off the tap for fossil carbon, Nova-Institut für politische und ökologische Innovation GmbH. 2021.
George S, Larsson G, Enfors SO. A scale-down two-compartment reactor with controlled substrate oscillations: metabolic response of Saccharomyces cerevisiae. Bioprocess Engineering. 1993 November; 9:249-57.
Lee SY, Kim HU. Systems strategies for developing industrial microbial strains. Nature biotechnology. 2015 October; 33(10):1061-72.
Limberg MH, Joachim M, Klein B, Wiechert W, Oldiges M. pH fluctuations imperil the robustness of C. glutamicum to short term oxygen limitation. Journal of biotechnology. 2017 Oct. 10; 259:248-60.
Liu Y, Nielsen J. Recent trends in metabolic engineering of microbial chemical factories. Current opinion in biotechnology. 2019 Dec. 1; 60:188-97.
Meyer HP, Schmidhalter DR. Microbial expression systems and manufacturing from a market and economic-perspective. Innovations in Biotechnology. Rijeka, Croatia, InTech. 2012 Feb 17:211-50.
Parekh S, Vinci VA, Strobel RJ. Improvement of microbial strains and fermentation processes. Applied microbiology and biotechnology. 2000 Sep.; 54:287-301.
Tobert JA. Lovastatin and beyond: the history of the HMG-CoA reductase inhibitors. Nature reviews Drug discovery. 2003 Jul. 1; 2(7):517-26.
Wehrs M, Tanjore D, Eng T, Lievense J, Pray TR, Mukhopadhyay A. Engineering robust production microbes for large-scale cultivation. Trends in microbiology. 2019 Jun. 1; 27(6):524-37.

The teachings of all patents, published applications and references cited herein are incorporated by reference in their entirety.

### EQUIVALENTS

While example embodiments have been particularly shown and described, it will be understood by those skilled in the art that various changes in form and details may be made therein without departing from the scope of the embodiments encompassed by the appended claims.

What is claimed is:

1. A bioreactor system comprising:
a chamber support assembly configured to mount a support rail, the support rail being configured to support a set of bioreactor chamber nodes;
a fluid manifold comprising a fill line fluid connection configured to permit filling of the set of bioreactor chamber nodes, and a drain line fluid connection configured to permit draining of the set of bioreactor chamber nodes; and
a chamber interface assembly comprising an optical guide configured to aim an optical fiber at an optical access window of a bioreactor chamber node of the set of bioreactor chamber nodes.

2. The bioreactor system of claim 1, further comprising the chamber support assembly, the chamber support assembly comprising a bioreactor chamber set lock with a notch configured to locate the set of bioreactor chamber nodes in position after the set is inserted into the chamber support assembly.

3. The bioreactor system of claim 1, wherein the chamber interface assembly further comprises:
a valve actuator configured to rotate a plurality of valves to pinch closed base tubing, the base tubing providing fluid connection between at least two bioreactor chamber nodes of the set of bioreactor chamber nodes.

4. The bioreactor system of claim 1, wherein the chamber interface assembly comprises an optical fiber for each bioreactor chamber node of the set of bioreactor chamber nodes.

5. The bioreactor system of claim 1, wherein the chamber interface assembly further comprises more than one optical fiber for each bioreactor chamber node of the set of bioreactor chamber nodes.

6. The bioreactor system of claim 1, wherein the chamber interface assembly comprises a linear actuator configured to position the optical guide relative to the optical access window of the bioreactor chamber node.

7. The bioreactor system of claim 1, wherein the chamber interface assembly further comprises a strain gauge configured to determine a weight of a bioreactor chamber node of the plurality of bioreactor chamber nodes.

8. The bioreactor system of claim 7, wherein the set of bioreactor chamber nodes is configured to be suspended from the support rail, and wherein the strain gauge is configured to sense a weight added to the bioreactor chamber node via additional load on the base of the bioreactor chamber node when the weight is added to the bioreactor chamber node.

9. The bioreactor system of claim 8, wherein the set of bioreactor chamber nodes is suspended from the support rail in a mechanically-compressed state.

10. The bioreactor system of claim 1, further comprising an optical multiplexer module configured for optical sensing of the plurality of bioreactor chamber nodes of the set of bioreactor chamber nodes.

11. The bioreactor system of claim 10, wherein the optical multiplexer module is configured to restrict sensing to one bioreactor chamber node of the plurality of bioreactor chamber nodes at a given time and is configured to change the one bioreactor chamber node to be sensed.

12. The bioreactor system of claim 10, wherein the optical multiplexer module comprises a linear stage to position an optical sensor relative to at least one optical fiber.

13. The bioreactor system of claim 1, further comprising at least one optical fiber configured to perform optical sensing of at least one bioreactor chamber node of the plurality of bioreactor chamber nodes.

14. The bioreactor system of claim 1, wherein the chamber interface assembly further comprises:
a guide rail configured to fit with an alignment rail of a bioreactor chamber node of the set of bioreactor chamber nodes;
a valve actuator configured to rotate a plurality of valves to pinch closed base tubing, the base tubing providing fluid connection between at least two bioreactor chamber nodes of the set of bioreactor chamber nodes; and
a strain gauge configured to determine a weight of the bioreactor chamber node of the plurality of bioreactor chamber nodes.

15. The bioreactor system of claim 1, further comprising:
a bioreactor chamber node of the set of bioreactor chamber nodes, the bioreactor chamber node comprising:
a flexible, oxygen-permeable membrane configured to contain a biotic or abiotic material for a bioreaction;
a rigid top cap mounted to a top portion of the flexible, oxygen-permeable membrane, the rigid top cap configured to mount to the support rail;
a rigid bottom cap mounted to a bottom portion of the flexible, oxygen-permeable membrane, the rigid bottom cap comprising at least one fluid access port; and
an optical access window configured to permit optical monitoring of the bioreaction by an optical fiber.

16. The bioreactor system of claim 1, wherein the fill line fluid connection and a plurality of fluid access ports of each of the bioreactor chamber nodes of the bioreactor system are connected to permit filling of all of the plurality of bioreactor chamber nodes of the bioreactor system in a single filling.

17. The bioreactor system of claim 1, further comprising a controlled environment containment surrounding the bioreactor system, the controlled environment containment comprising a gas surrounding the bioreactor system.

18. The bioreactor system of claim 1, further comprising a system fluid manifold that distributes a single fluidic fill path to a plurality of sets of the bioreactor chamber nodes, or that collects a single fluidic drain path from a plurality of sets of the bioreactor chamber nodes.

19. A bioreactor system comprising:
a chamber support assembly configured to mount a support rail, the support rail configured to support a set of bioreactor chamber nodes;
a fluid manifold comprising a fill line fluid connection configured to permit filling of the set of bioreactor chamber nodes, and a drain line fluid connection configured to permit draining of the set of bioreactor chamber nodes; and
a bioreactor chamber node of the set of bioreactor chamber nodes, the bioreactor chamber node comprising:
a flexible, oxygen-permeable membrane configured to contain a biotic or abiotic material for a bioreaction;
a rigid top cap mounted to a top portion of the flexible, oxygen-permeable membrane, the rigid top cap configured to mount to the support rail;
a rigid bottom cap mounted to a bottom portion of the flexible, oxygen-permeable membrane, the rigid bottom cap comprising at least one fluid access port; and
an optical access window configured to permit optical monitoring of the bioreaction by an optical fiber.

20. The bioreactor system of claim 19, wherein the bioreactor chamber node further comprises:
an alignment rail configured to align the bioreactor chamber node with a guide rail of a chamber interface assembly of the bioreactor system.

21. The bioreactor system of claim 19, wherein the bioreactor chamber node is configured to be suspended from the support rail.

22. The bioreactor system of claim 21, wherein the bioreactor chamber node is configured to be suspended from the support rail in a mechanically compressed state.

23. The bioreactor system of claim 19, wherein the at least one fluid access port comprises a fill port and a dispense port.

24. The bioreactor system of claim 23, further comprising:
a fill connection barb, in fluid connection with the fill port, configured to provide fluid connection to fill base tubing of the bioreactor system; and
a dispense connection barb in fluid connection with the dispense port, configured to provide fluid connection to dispense base tubing of the bioreactor system.

25. The bioreactor system of claim 19, further comprising a plurality of valves configured to restrict fluid flow to or from the bioreactor chamber nodes.

26. The bioreactor system of claim 19, wherein the fill line fluid connection and a plurality of fluid access ports of the bioreactor chamber nodes are connected to permit filling of all bioreactor chamber nodes of the set of bioreactor chamber nodes in a single filling.

27. The bioreactor system of claim 19, wherein the bioreactor chamber node is a disposable module that is configured to be used once and then disposed of.

28. The bioreactor system of claim 19, further comprising a controlled environment containment surrounding the bioreactor system, the controlled environment containment comprising a gas surrounding the bioreactor system.

29. A bioreactor chamber assembly for use in a bioreactor system, the bioreactor chamber assembly comprising:
a support rail supporting a set of a plurality of bioreactor chamber nodes, the plurality of bioreactor chamber nodes being configured to be suspended from the support rail;
each bioreactor chamber node of the plurality of bioreactor chamber nodes comprising:
a flexible, oxygen-permeable membrane configured to contain a biotic or abiotic material for a bioreaction;
a rigid top cap mounted to a top portion of the flexible, oxygen-permeable membrane, the rigid top cap configured to mount to the support rail;
a rigid bottom cap mounted to a bottom portion of the flexible, oxygen-permeable membrane, the rigid bottom cap comprising at least one fluid access port; and
an optical access window configured to permit optical monitoring of the bioreaction by an optical fiber.

30. A bioreactor chamber node for a bioreactor system, the bioreactor chamber node comprising:
a flexible, oxygen-permeable membrane configured to contain a biotic or abiotic material for a bioreaction;
a rigid top cap mounted to a top portion of the flexible, oxygen-permeable membrane, the rigid top cap configured to mount to a support rail of a bioreactor chamber assembly;
a rigid bottom cap mounted to a bottom portion of the flexible, oxygen-permeable membrane, the rigid bottom cap comprising at least one fluid access port; and
an optical access window configured to provide optical monitoring of the bioreaction by an optical fiber.
